(19) United States
(12) Patent Application Publication
(10) Pub. No.: US 2006/0264424 A1
(43) Pub. Date: Nov. 23, 2006

(54) Arylsulfonamidobenzylic compounds

(75) Inventors: Martin J. Harrison, Stockport (GB); Xian Yun Jiao, San Mateo, CA (US); Stuart D. Jones, Prestbury (GB); Frank Kayser, San Francisco, CA (US); David J. Kopecky, San Francisco, CA (US)

Correspondence Address: AMGEN INC., 1120 VETERANS BOULEVARD, SOUTH SAN FRANCISCO, CA, 94080, US

(73) Assignee: Tularik Inc, South San Francisco, CA (US)

(21) Appl. No.: 10/916,868

(22) Filed: Aug. 11, 2004

Related U.S. Application Data
(60) Provisional application No. 60/494,692, filed on Aug. 12, 2003.

(51) Int. Cl. A61K 31/54; A61K 31/537; A61K 31/495; A61K 31/445; A61K 31/40

(52) U.S. Cl. 514/227.5; 514/237.5; 514/252.12; 514/317; 514/408; 544/59; 544/158; 544/399; 546/229

(57) ABSTRACT

Compounds, pharmaceutical compositions and methods are provided that are useful in the treatment or prevention of lipid disorders, metabolic disorders and cell-proliferative diseases. In particular, the invention provides compounds which modulate the expression and/or function of proteins involved in cholesterol metabolism. The subject compounds are particularly useful in the treatment of obesity, diabetes, hypercholesterolemia, atherosclerosis and hypolipoproteinemia.

### CROSS-REFERENCES TO RELATED APPLICATIONS

This application is related to and claims the benefit of U.S. Application Ser. No. 60/494,692, filed Aug. 12, 2003, the disclosure of which is incorporated by reference herein. This application is related to U.S. patent application Ser. No. 10/354,922, filed Jan. 29, 2003, and U.S. patent application Ser. No. 10/354,923, filed Jan. 29, 2003, the disclosures of which are incorporated herein by reference.

### BACKGROUND OF THE INVENTION

Cholesterol is used for the synthesis of bile acids in the liver, the manufacture and repair of cell membranes, and the synthesis of steroid hormones. There are both exogenous and endogenous sources of cholesterol. The average American consumes about 450 mg of cholesterol each day and produces an additional 500 to 1,000 mg in the liver and other tissues. Another source is the 500 to 1,000 mg of biliary cholesterol that is secreted into the intestine daily; about 50 percent is reabsorbed (enterohepatic circulation). Excess accumulation of cholesterol in the arterial walls can result in atherosclerosis, which is characterized by plaque formation. The plaques inhibit blood flow, promote clot formation and can ultimately cause heart attacks, stroke and claudication. Development of therapeutic agents for the treatment of atherosclerosis and other diseases associated with cholesterol metabolism has been focused on achieving a more complete understanding of the biochemical pathways involved. Most recently, liver X receptors (LXRs) were identified as key components in cholesterol homeostasis.

The LXRs were first identified as orphan members of the nuclear receptor superfamily whose ligands and functions were unknown. Two LXR proteins (α and β) are known to exist in mammals. The expression of LXRα is restricted, with the highest levels being found in the liver and lower levels found in kidney, intestine, spleen, and adrenals (see Willy et al. (1995) Genes Dev. 9(9):1033-1045). LXRβ is rather ubiquitous, being found in nearly all tissues examined. Recent studies on the LXRs indicate that they are activated by certain naturally occurring, oxidized derivatives of cholesterol, including 22(R)-hydroxycholesterol, 24(S)-hydroxycholesterol and 24,25(S)-epoxycholesterol (see Lehmann et al. (1997) J. Biol. Chem. 272(6):3137-3140). The expression pattern of LXRs and their oxysterol ligands provided the first hint that these receptors may play a role in cholesterol metabolism (see Janowski et al. (1996) Nature 383:728-731).

As noted above, cholesterol metabolism in mammals occurs via conversion into steroid hormones or bile acids. The role of LXRs in cholesterol homeostasis was first postulated to involve the pathway of bile acid synthesis, in which cholesterol 7α-hydroxylase (CYP7A) operates in a rate-limiting manner. Support for this proposal was provided when additional experiments found that the CYP7A promoter contained a functional LXR response element that could be activated by RXR/LXR heterodimers in an oxysterol- and retinoid-dependent manner. Confirmation of LXR function as a transcriptional control point in cholesterol metabolism was made using knockout mice, particularly those lacking the oxysterol receptor LXRα (see Peet et al. (1998) Cell 93:693-704).

Mice lacking the receptor LXRα (e.g., knockout or (−/−) mice) lost their ability to respond normally to increases in dietary cholesterol and were unable to tolerate any cholesterol in excess of that synthesized de novo. LXRα (−/−) mice did not induce transcription of the gene encoding CYP7A when fed diets containing additional cholesterol. This resulted in an accumulation of large amounts of cholesterol and impaired hepatic function in the livers of LXRα (−/−) mice. These results further established the role of LXRα as the essential regulatory component of cholesterol homeostasis. LXRα is also believed to be involved in fatty acid synthesis. Accordingly, regulation of LXRα (e.g., use of LXRα agonist or antagonists) could provide treatment for a variety of lipid disorders including obesity and diabetes.

In view of the importance of LXRs, and particularly LXRα, to the delicate balance of cholesterol metabolism and fatty acid biosynthesis, we describe modulators of LXRs which are useful as therapeutic agents or diagnostic agents for the treatment of disorders associated with bile acid and cholesterol metabolism, including cholesterol gallstones, atherosclerosis, lipid storage diseases, obesity and diabetes. The agents described herein are also useful for disease states associated with serum hypercholesterolemia, such as coronary heart disease.

### BRIEF SUMMARY OF THE INVENTION

In one aspect, the present invention provides compounds having the formula: 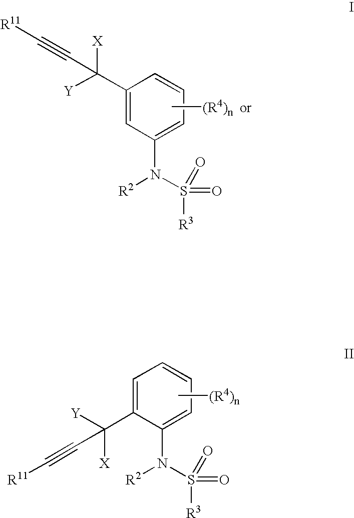
wherein R¹¹ is a member selected from the group consisting of hydrogen, halogen, nitro, cyano, R¹², OR¹², SR¹², NHR¹², N(R¹²)₂, (C₅-C₈)cycloalkenyl, COR¹², CO₂R¹², CONHR¹², CON(R¹²)₂, C═N—NR¹², aryl(C₁-C₄)alkyl, heteroaryl, heteroaryl(C₁-C₄)alkyl, (C₄-C₈)cycloalkyl(C₁-C₄)alkyl and hetero(C₄-C₈)cycloalkyl(C₁-C₄)alkyl; wherein each R¹² is (C₁-C₈)alkyl, (C₃-C₈)alkenyl, (C₃-C₈)alkynyl, (C₂-C₈)heteroalkyl, halo(C₁-C₈)alkyl, (C₄-C₈)cycloalkyl, aryl or two R¹² groups attached to the same nitrogen atom are combined to form a five- to eight-membered ring and any alkyl portions of R¹¹ are optionally substituted with from one to three substituents independently selected from the group consisting of halogen, OR¹³, NHSO₂R¹⁴ and NHC(O)R¹³, and any aryl or heteroaryl portions of R¹¹ are optionally substituted with from one to five substituents independently selected from the group consisting of halogen, cyano, nitro, R¹⁴, OR¹³, SR¹³, N(R¹³)₂, CO₂R¹³, CON(R¹³)₂, C(O)R¹³, SO₂R¹³, SO₂N(R¹³)₂, NHSO₂R¹⁴, NHC(O)R¹³, phenyl, phenyl(C₁-C₈)alkyl and phenyl(C₂-C₈)heteroalkyl; wherein each R¹³ is independently selected from H, (C₁-C₈)alkyl, (C₃-C₈)alkenyl, (C₃-C₈)alkynyl, (C₂-C₈)heteroalkyl and halo(C₁-C₈)alkyl and each R¹⁴ is independently selected from (C₁-C₈)alkyl, (C₃-C₈)alkenyl, (C₃-C₈)alkynyl, (C₂-C₈)heteroalkyl and halo(C₁-C₈)alkyl.

X represents H, NH₂, NHR¹⁵, NHSO₂R¹⁵, OH or OR′, wherein R¹⁵ is (C₁-C₈)alkyl, (C₃-C₈)alkenyl, (C₃-C₈)alkynyl, (C₂-C₈)heteroalkyl or halo(C₁-C₈)alkyl and R′ is (C₁-C₈)alkyl, (C₃-C₈)alkenyl, (C₃-C₈)alkynyl, (C₂-C₈)heteroalkyl, halo(C₁-C₈)alkyl, aryl(C₁-C₄)alkyl, heterocyclo(C₅-C₈)alkyl, (C₁-C₄)alkylsulfonyl, arylsulfonyl, (C₁-C₄)alkylcarbonyl or (C₁-C₄)alkylsilyl; and Y is fluoro(C₁-C₄)alkyl.

R² is selected from H, (C₁-C₈)alkyl, (C₂-C₈)heteroalkyl, (C₃-C₈)alkenyl, (C₃-C₈)alkynyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino; and R³ is selected from aryl and heteroaryl, the aryl or heteroaryl group being optionally substituted with from one to five substituents independently selected from halogen, cyano, nitro, R¹⁶, OR¹⁶, SR¹⁶, COR¹⁶, CO₂R¹⁶, NHR¹⁶, N(R¹⁶)₂, CONHR¹⁶, CON(R¹⁶)₂, NHSO₂R¹⁶, NHC(O)R¹⁶, phenyl, phenyl(C₁-C₈)alkyl and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring. Optionally, R² and R⁴ are combined to form a five- to seven-membered fused ring containing the nitrogen atom to which R² is attached and from 0 to 2 additional heteroatoms selected from N, O and S.

The subscript n is an integer of from 0 to 3, indicating the presence or absence of substituents on the phenyl ring core of formulas I and II. Each of the R⁴ substituents is independently selected from halogen, cyano, nitro, R¹⁷, OR¹⁷, SR¹⁷, COR¹⁷, CO₂R¹⁷, N(R¹⁷)₂ and CON(R¹⁷)₂, wherein each R¹⁷ is independently selected from H, (C₁-C₈)alkyl, (C₃-C₈)alkenyl, (C₃-C₈)alkynyl, (C₂-C₈)heteroalkyl or halo(C₁-C₈)alkyl, or two R¹⁷ groups attached to the same nitrogen atom are combined to form a five- to eight-membered ring.

In addition to the compounds provided in formulas I and II, pharmaceutically acceptable salts and prodrugs thereof are also provided.

In yet another aspect, the present invention provides methods for modulating LXR in a cell by administering to or contacting the cell with a composition containing a compound of formula I or II above.

In still another aspect, the present invention provides methods for treating LXR-responsive diseases by administering to a subject in need of such treatment a composition containing a compound of formula I or II. These methods are particularly useful for the treatment of pathology such as obesity, diabetes, hypercholesterolemia, atherosclerosis and hyperlipoproteinemia. In certain embodiments, the compound can be administered to the subject in combination with an additional anti-hypercholesterolemic agent, for example, bile acid sequestrants, nicotinic acid, fibric acid derivatives or HMG CoA reductase inhibitors.

The present compounds can exert their effects either systemically (the compounds permeate the relevant tissues, such as liver, upon entrance into the bloodstream) or locally (for example, by modulating LXR function of intestinal epithelial cells following oral administration, without necessitating the compounds' entrance into the bloodstream). In some disease states, some preferred compounds will be those with good systemic distribution, while, in other instances, preferred compounds will be those that can work locally on the intestinal track or on the skin without penetrating the bloodstream.

Certain compounds of the present invention are antiproliferative and can be used in compositions for treating diseases associated with abnormal cell proliferation (e.g., cancer). Other diseases associated with an abnormally high level of cellular proliferation include restenosis, where vascular smooth muscle cells are involved, inflammatory disease states, where endothelial cells, inflammatory cells and glomerular cells are involved, myocardial infarction, where heart muscle cells are involved, glomerular nephritis, where kidney cells are involved, transplant rejection, where endothelial cells are involved, infectious diseases such as HIV infection and malaria, where certain immune cells and/or other infected cells are involved, and the like. Infectious and parasitic agents per se (e.g. bacteria, trypanosomes, fungi, etc.) are also subject to selective proliferative control using the subject compositions and compounds.

### BRIEF DESCRIPTION OF THE DRAWINGS

Not applicable.

### DETAILED DESCRIPTION OF THE INVENTION

Definitions

As used herein, the term “heteroatom” is meant to include oxygen (O), nitrogen (N), sulfur (S) and silicon (Si).

The term “alkyl,” by itself or as part of another substituent, means, unless otherwise stated, a straight or branched chain, or cyclic hydrocarbon radical, or combination thereof, which is fully saturated, having the number of carbon atoms designated (i.e. C₁-C₈ means one to eight carbons). Examples of alkyl groups include methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl and the like.

The term “alkenyl”, by itself or as part of another substituent, means a straight or branched chain, or cyclic hydrocarbon radical, or combination thereof, which may be mono- or polyunsaturated, having the number of carbon atoms designated (i.e. C₃-C₈ means three to eight carbons) and one or more double bonds. Examples of alkenyl groups include vinyl, 2-propenyl, crotyl, 2-isopentenyl, 2-(butadienyl), 2,4-pentadienyl, 3-(1,4-pentadienyl) and higher homologs and isomers thereof.

The term “alkynyl”, by itself or as part of another substituent, means a straight or branched chain hydrocarbon radical, or combination thereof, which may be mono- or polyunsaturated, having the number of carbon atoms designated (i.e. C₃-C₈ means three to eight carbons) and one or more triple bonds. Examples of alkynyl groups include ethynyl, 1- and 3-propynyl, 3-butynyl and higher homologs and isomers thereof.

The term “alkylene” by itself or as part of another substituent means a divalent radical derived from alkyl, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃.

Similarly, the term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Accordingly, a cycloalkyl group has the number of carbon atoms designated (i.e., C₃-C₈ means three to eight carbons) and may also have one or two double bonds. A heterocycloalkyl group consists of the number of carbon atoms designated and from one to three heteroatoms selected from the group consisting of O, N, Si and S, and wherein the nitrogen and sulfur atoms may optionally be oxidized and the nitrogen heteroatom may optionally be quaternized. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” and “halogen,” by themselves or as part of another substituent, mean, unless otherwise stated, a fluorine, chlorine, bromine, or iodine atom. Additionally, terms such as “haloalkyl,” are meant to include alkyl substituted with halogen atoms, which can be the same or different, in a number ranging from one to (2 m′+1), where m′ is the total number of carbon atoms in the alkyl group. For example, the term “halo(C₁-C₄)alkyl” is mean to include trifluoromethyl, 2,2,2-trifluoroethyl, 4-chlorobutyl, 3-bromopropyl, and the like. Thus, the term “haloalkyl” includes monohaloalkyl (alkyl substituted with one halogen atom) and polyhaloalkyl (alkyl substituted with halogen atoms in a number ranging from two to (2 m′+1) halogen atoms, where m′ is the total number of carbon atoms in the alkyl group). The term “perhaloalkyl” means, unless otherwise stated, alkyl substituted with (2 m′+1) halogen atoms, where m′ is the total number of carbon atoms in the alkyl group. For example the term “perhalo(C₁-C₄)alkyl” is meant to include trifluoromethyl, pentachloroethyl, 1,1,1-trifluoro-2-bromo-2-chloroethyl and the like.

The term “acyl” refers to those groups derived from an organic acid by removal of the hydroxy portion of the acid. Accordingly, acyl is meant to include, for example, acetyl, propionyl, butyryl, decanoyl, pivaloyl, benzoyl and the like.

The term “aryl” means, unless otherwise stated, a polyunsaturated, typically aromatic, hydrocarbon substituent which can be a single ring or multiple rings (up to three rings) which are fused together or linked covalently. Non-limiting examples of aryl groups include phenyl, 1-naphthyl, 2-naphthyl, 4-biphenyl and 1,2,3,4-tetrahydronaphthalene.

The term “heteroaryl” refers to aryl groups (or rings) that contain from zero to four heteroatoms selected from N, O, and S, wherein the nitrogen and sulfur atoms are optionally oxidized and the nitrogen heteroatom are optionally quaternized. A heteroaryl group can be attached to the remainder of the molecule through a heteroatom. Non-limiting examples of heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 2-pyrimidinyl, 4-pyrimidinyl, 5-pyrimidinyl, 3-pyridazinyl, 4-pyridazinyl, 5-benzothiazolyl, purinyl, 2-benzimidazolyl, 5-indolyl, 1H-indazolyl, carbazolyl, α-carbolinyl, β-carbolinyl, γ-carbolinyl, 1-isoquinolyl, 5-isoquinolyl, 2-quinoxalinyl, 5-quinoxalinyl, 2-quinolyl, 3-quinolyl, 4-quinolyl, 5-quinolyl, 6-quinolyl, 7-quinolyl and 8-quinolyl.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) is meant to include both substituted and unsubstituted forms of the indicated radical, unless otherwise indicated. Preferred substituents for each type of radical are provided below.

Substituents for the alkyl and heteroalkyl radicals (as well as those groups referred to as alkylene, alkenyl, heteroalkylene, heteroalkenyl, alkynyl, cycloalkyl, heterocycloalkyl, cycloalkenyl and heterocycloalkenyl) can be a variety of groups selected from: —OR′, ═O, ═NR′, ═N—OR′, —NR′R″, —SR′, halogen, —SiR′R″R′″, —OC(O)R′, —C(O)R′, —CO₂R′, —CONR′ R″, —OC(O)NR′ R″, —NR″C(O)R′, —NR′—C(O)NR″R′″, —NR′—SO₂NR″R′″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN and —NO₂, in a number ranging from zero to three, with those groups having zero, one or two substituents being particularly preferred. R′, R″ and R′″ each independently refer to hydrogen, unsubstituted (C₁-C₈)alkyl and heteroalkyl, unsubstituted aryl, aryl substituted with one to three halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6- or 7-membered ring. For example, —NR′R″ is meant to include 1-pyrrolidinyl and 4-morpholinyl. Typically, an alkyl or heteroalkyl group will have from zero to three substituents, with those groups having two or fewer substituents being preferred in the present invention. More preferably, an alkyl or heteroalkyl radical will be unsubstituted or monosubstituted. Most preferably, an alkyl or heteroalkyl radical will be unsubstituted. From the above discussion of substituents, one of skill in the art will understand that the term “alkyl” is meant to include groups such as trihaloalkyl (e.g., —CF₃ and —CH₂CF₃).

Preferred substituents for the alkyl and heteroalkyl radicals are selected from: —OR′, ═O, —NR′R″, —SR′, halogen, —SiR′R″R′″, —OC(O)R′, —C(O)R′, —CO₂R′, —CONR′R″, —OC(O)NR′R″, —NR″C(O)R′, —NR″CO₂R′, —NR′—SO₂NR″R′″, —S(O)R′, —SO₂R′, —SO₂NR′R″, —NR″SO₂R, —CN and —NO₂, where R′ and R″ are as defined above. Further preferred substituents are selected from: —OR′, ═O, —NR′R″, halogen, —OC(O)R′, —CO₂R′, —CONR′R″, —OC(O)NR′R″, —NR″C(O)R′, —NR″CO₂R′, —NR′—SO₂NR″R′″, —SO₂R′, —SO₂NR′R″, —NR″SO₂R, —CN and —NO₂.

Similarly, substituents for the aryl and heteroaryl groups are varied and selected from: halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″CO₂R′, —NR′—C(O)NR″R′″, —NR′—SO₂NR″R′″, —NH—C(NH₂)═NH, —NR′C(NH₂)═NH, —NH—C(NH₂)═NR′, —S(O)R′, —SO₂R′, —SO₂NR′R″, —NR″SO₂R, —N₃, —CH(Ph)₂, perfluoro(C₁-C₄)alkoxy and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl and (unsubstituted aryl)oxy-(C₁-C₄)alkyl. When the aryl group is 1,2,3,4-tetrahydronaphthalene, it may be substituted with a substituted or unsubstituted (C₃-C₇)spirocycloalkyl group. The (C₃-C₇)spirocycloalkyl group may be substituted in the same manner as defined herein for “cycloalkyl”. Typically, an aryl or heteroaryl group will have from zero to three substituents, with those groups having two or fewer substituents being preferred in the present invention. In one embodiment of the invention, an aryl or heteroaryl group will be unsubstituted or monosubstituted. In another embodiment, an aryl or heteroaryl group will be unsubstituted.

Preferred substituents for aryl and heteroaryl groups are selected from: halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —S(O)R′, —SO₂R′, —SO₂NR′R″, —NR″SO₂R, —N₃, —CH(Ph)₂, perfluoro(C₁-C₄)alkoxy and perfluoro(C₁-C₄)alkyl, where R′ and R″ are as defined above. Further preferred substituents are selected from: halogen, —OR′, —OC(O)R′, —NR′R″, —R′, —CN, —NO₂, —CO₂R′, —CONR′R″, —NR″C(O)R′, —SO₂R′, —SO₂NR′R″, —NR″SO₂R, perfluoro(C₁-C₄)alkoxy and perfluoro(C₁-C₄)alkyl.

It is to be understood that the substituent —CO₂H, as used herein, includes bioisosteric replacements therefor, such as: 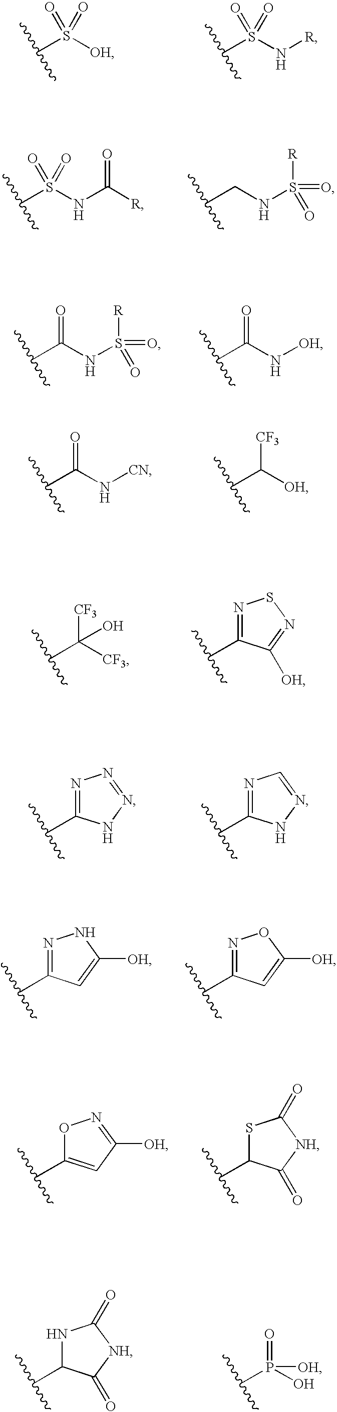
and the like. See, e.g., The Practice of Medicinal Chemistry; Wermuth, C. G., Ed.; Academic Press: New York, 1996; p. 203.

Two of the substituents on adjacent atoms of the aryl or heteroaryl ring may optionally be replaced with a substituent of the formula -T-C(O)—(CH₂)_{q}-U-, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic and the like. Also included are salts of amino acids such as arginate and the like, and salts of organic acids like glucuronic or galactunoric acids and the like (see, for example, Berge et al. (1977) J. Pharm. Sci. 66:1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, for instance, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The terms “modulate”, “modulation” and the like refer to the ability of a compound to increase or decrease the function and/or expression of LXR, where LXR function may include transcription regulatory activity and/or protein-binding. Modulation may occur in vitro or in vivo. Modulation, as described herein, includes antagonism, agonism, partial antagonism and/or partial agonism of a function or characteristic associated with LXR, either directly or indirectly, and/or the upregulation or downregulation of LXR expression, either directly or indirectly. Agonists are compounds that, e.g., bind to, stimulate, increase, open, activate, facilitate, enhance activation, activate, sensitize or upregulate signal transduction. Antagonists are compounds that, e.g., bind to, partially or totally block stimulation, decrease, prevent, inhibit, delay activation, inactivate, desensitize, or downregulate signal transduction. A modulator preferably inhibits LXR function and/or downregulates LXR expression. More preferably, a modulator inhibits or activates LXR function and/or downregulates or upregulates LXR expression. Most preferably, a modulator activates LXR function and/or upregulates LXR expression. The ability of a compound to modulate LXR function can be demonstrated in a binding assay or a cell-based assay, e.g., a transient transfection assay.

As used herein, “diabetes” refers to type I diabetes mellitus (juvenile onset diabetes, insulin dependent-diabetes mellitus or IDDM) or type II diabetes mellitus (non-insulin-dependent diabetes mellitus or NIDDM), preferably, NIDDM.

As used herein, the term “LXR-mediated condition or disorder” refers to a condition or disorder characterized by inappropriate, e.g., less than or greater than normal, LXR activity. Inappropriate LXR functional activity might arise as the result of LXR expression in cells which normally do not express LXR, decreased LXR expression (leading to, e.g., lipid and metabolic disorders and diseases) or increased LXR expression. An LXR-mediated condition or disease may be completely or partially mediated by inappropriate LXR functional activity. However, an LXR-mediated condition or disease is one in which modulation of LXR results in some effect on the underlying condition or disorder (e.g., an LXR agonist results in some improvement in patient well-being in at least some patients).

As used herein, the term “LXR-responsive condition” or “LXR-responsive disorder” refers to a condition or disorder that responds favorably to modulation of LXR activity. Favorable responses to LXR modulation include alleviation or abrogation of the disease and/or its attendant symptoms, inhibition of the disease, i.e., arrest or reduction of the development of the disease, or its clinical symptoms, and regression of the disease or its clinical symptoms. An LXR-responsive condition or disease may be completely or partially responsive to LXR modulation. An LXR-responsive condition or disorder may be associated with inappropriate, e.g., less than or greater than normal, LXR activity. Inappropriate LXR functional activity might arise as the result of LXR expression in cells which normally do not express LXR, decreased LXR expression (leading to, e.g., lipid and metabolic disorders and diseases) or increased LXR expression. An LXR-responsive condition or disease may include an LXR-mediated condition or disease.

The term “therapeutically effective amount” refers to the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

General

The present invention provides compositions, compounds and methods for modulating LXR function in a cell. The compositions which are useful for this modulation will typically be those which contain an effective amount of an LXR-modulating compound. In general, an effective amount of an LXR-modulating compound is a concentration of the compound that will produce at 50 percent increase/decrease in LXR activity in a cell-based reporter gene assay, or a biochemical peptide-sensor assay such as the assays described in U.S. Pat. No. 6,555,326 and U.S. patent application Ser. No. 09/163,713 (filed Sep. 30, 1998).

### EMBODIMENTS OF THE INVENTION

Compounds

In one aspect, the present invention provides compounds having the formula: 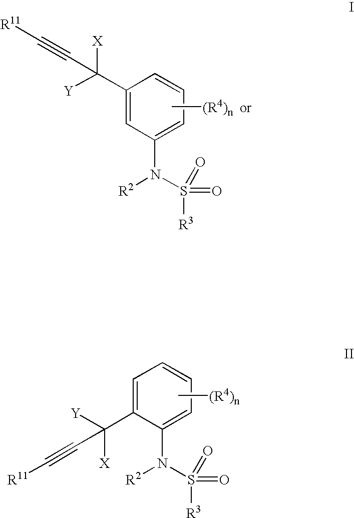
wherein R¹¹ is selected from hydrogen, halogen, nitro, cyano, R¹², OR¹², SR¹², NHR¹², N(R¹²)₂, (C₅-C₈)cycloalkenyl, COR¹², CO₂R¹², CONHR¹², CON(R¹²)₂, C═N—NR¹², aryl(C₁-C₄)alkyl, heteroaryl, heteroaryl(C₁-C₄)alkyl, (C₄-C₈)cycloalkyl(C₁-C₄)alkyl and hetero(C₄-C₈)cycloalkyl(C₁-C₄)alkyl; wherein each R¹² is (C₁-C₈)alkyl, (C₃-C₈)alkenyl, (C₃-C₈)alkynyl, (C₂-C₈)heteroalkyl, halo(C₁-C₈)alkyl, (C₄-C₈)cycloalkyl, aryl or two R¹² groups attached to the same nitrogen atom are combined to form a five- to eight-membered ring and any alkyl portions of R¹¹ are optionally substituted with from one to three substituents independently selected from halogen, OR¹³, NHSO₂R¹⁴ and NHC(O)R¹³, and any aryl or heteroaryl portions of R¹¹ are optionally substituted with from one to five substituents independently selected from halogen, cyano, nitro, R¹⁴, OR¹³, SR¹³, N(R¹³)₂, CO₂R¹³, CON(R¹³)₂, C(O)R¹³, SO₂R¹³, SO₂N(R¹³)₂, NHSO₂R¹⁴, NHC(O)R¹³, phenyl, phenyl(C₁-C₈)alkyl and phenyl(C₂-C₈)heteroalkyl; wherein each R¹³ is independently selected from H, (C₁-C₈)alkyl, (C₃-C₈)alkenyl, (C₃-C₈)alkynyl, (C₂-C₈)heteroalkyl and halo(C₁-C₈)alkyl and each R¹⁴ is independently selected from (C₁-C₈)alkyl, (C₃-C₈)alkenyl, (C₃-C₈)alkynyl, (C₂-C₈)heteroalkyl and halo(C₁-C₈)alkyl.

X represents H, NH₂, NHR¹⁵, NHSO₂R¹⁵, OH or OR′, wherein R¹⁵ is (C₁-C₈)alkyl, (C₃-C₈)alkenyl, (C₃-C₈)alkynyl, (C₂-C₈)heteroalkyl or halo(C₁-C₈)alkyl and R′ is (C₁-C₈)alkyl, (C₃-C₈)alkenyl, (C₃-C₈)alkynyl, (C₂-C₈)heteroalkyl, halo(C₁-C₈)alkyl, aryl(C₁-C₄)alkyl, heterocyclo(C₅-C₈)alkyl, (C₁-C₄)alkylsulfonyl, arylsulfonyl, (C₁-C₄)alkylcarbonyl or (C₁-C₄)alkylsilyl; and Y is fluoro(C₁-C₄)alkyl. In particularly preferred embodiments, Y is CF₃.

R² is selected from H, (C₁-C₈)alkyl, (C₂-C₈)heteroalkyl, (C₃-C₈)alkenyl, (C₃-C₈)alkynyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino; and R³ is selected from aryl and heteroaryl, the aryl or heteroaryl group being optionally substituted with from one to five substituents independently selected from halogen, cyano, nitro, R¹⁶, OR¹⁶, SR¹⁶, COR¹⁶, CO₂R¹⁶, NHR¹⁶, N(R¹⁶)₂, CONHR¹⁶, CON(R¹⁶)₂, NHSO₂R¹⁶, NHC(O)R¹⁶, phenyl, phenyl(C₁-C₈)alkyl, and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring.

The subscript n is an integer of from 0 to 3, indicating the presence or absence of substituents on the phenyl ring core of formulas I and II. Each of the R⁴ substituents is independently selected from halogen, cyano, nitro, R¹⁷, OR¹⁷, SR¹⁷, COR¹⁷, CO₂R¹⁷, N(R¹⁷)₂ and CON(R¹⁷)₂, wherein each R¹⁷ is independently selected from H, (C₁-C₈)alkyl, (C₃-C₈)alkenyl, (C₃-C₈)alkynyl, (C₂-C₈)heteroalkyl or halo(C₁-C₈)alkyl, or two R¹⁷ groups attached to the same nitrogen atom are combined to form a five- to eight-membered ring.

Also included in this aspect of the invention are any pharmaceutically acceptable salts or prodrugs of the above compounds.

In one group of preferred embodiments, X is H or X is OH.

In another group of preferred embodiments, R¹¹ is selected from phenyl, pyridyl, pyridazinyl, pyrimidinyl, imidazolyl, thienyl, thiazolyl, oxazolyl, pyrrolyl, pyrazolyl, tetrazolyl, indolyl, benzimidazolyl, benzothienyl and benzothiazolyl, each of these R¹¹ groups being optionally substituted with from one to five substituents independently selected from halogen, cyano, nitro, (C₁-C₈)alkyl, (C₃-C₈)alkenyl, (C₃-C₈)alkynyl, (C₂-C₈)heteroalkyl, halo(C₁-C₈)alkyl, phenyl(C₁-C₆)alkyl, phenyl(C₂-C₆)heteroalkyl and (C₁-C₄)alkylsulfonyl. In particularly preferred embodiments, Y is CF₃.

In still further preferred embodiments, R¹¹ is phenyl optionally substituted with from one to two substituents independently selected from the group consisting of halogen, cyano, nitro, (C₁-C₈)alkyl, (C₃-C₈)alkenyl, (C₃-C₈)alkynyl, (C₂-C₈)heteroalkyl, halo(C₁-C₈)alkyl, phenyl(C₁-C₆)alkyl, phenyl(C₂-C₆)heteroalkyl and (C₁-C₄)alkylsulfonyl.

R², R³ and R⁴ also have certain preferred members. In particular, R² is preferably selected from H, (C₁-C₈)alkyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino. R³ is preferably selected from phenyl, pyridyl, thienyl and thiazolyl, optionally substituted with from one to five substituents independently selected from the group consisting of halogen, cyano, nitro, R¹⁶, OR¹⁶, SR¹⁶, COR¹⁶, CO₂R¹⁶, NHR¹⁶, N(R¹⁶)₂, CONHR¹⁶, CON(R¹⁶)₂, NHSO₂R¹⁶, NHC(O)R¹⁶, phenyl, phenyl(C₁-C₈)alkyl, and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring. The subscript n is preferably 0, 1, or 2 and each R⁴ is preferably selected from halogen, (C₁-C₈)alkyl and halo(C₁-C₈)alkyl.

In another group of still further preferred embodiments, R¹¹ is pyrazolyl optionally substituted with from one to two substituents independently selected from halogen, cyano, nitro, (C₁-C₈)alkyl, (C₃-C₈)alkenyl, (C₃-C₈)alkynyl, (C₂-C₈)heteroalkyl, halo(C₁-C₈)alkyl, phenyl(C₁-C₆)alkyl, phenyl(C₂-C₆)heteroalkyl and (C₁-C₄)alkylsulfonyl. Preferred members of the remaining groups R², R³ and R⁴ are the same as have been described above for the embodiments in which R¹¹ is phenyl.

In yet another group of still further preferred embodiments, R¹¹ is thienyl optionally substituted with from one to two substituents independently selected from halogen, cyano, nitro, (C₁-C₈)alkyl, (C₃-C₈)alkenyl, (C₃-C₈)alkynyl, (C₂-C₈)heteroalkyl, halo(C₁-C₈)alkyl, phenyl(C₁-C₆)alkyl, phenyl(C₂-C₆)heteroalkyl and (C₁-C₄)alkylsulfonyl. Preferred members of the remaining groups R², R³ and R⁴ are the same as have been described above for the embodiments in which R¹¹ is phenyl.

The most preferred compounds of the present invention are those provided in the Examples below.

Some of the compounds of formula I or II may exist as stereoisomers, and the invention includes all active stereoisomeric forms of these compounds. In the case of optically active isomers, such compounds may be obtained from corresponding optically active precursors using the procedures described herein or by resolving racemic mixtures. The resolution may be carried out using various techniques such as chromatography, repeated recrystallization of derived asymmetric salts, or derivatization, which techniques are well known to those of ordinary skill in the art.

The compounds of the invention may be labeled in a variety of ways. For example, the compounds may contain radioactive isotopes such as, for example, ³H (tritium) and ¹⁴C (carbon-14). Similarly, the compounds may be advantageously joined, covalently or noncovalently, directly or through a linker molecule, to a wide variety of other compounds, which may provide prodrugs or function as carriers, labels, adjuvants, coactivators, stabilizers, etc. Such labeled and joined compounds are contemplated within the present invention.

In another aspect of the invention, pharmaceutical compositions are provided in which a compound of formula I or II is combined with a pharmaceutically acceptable carrier or diluent. Particular compositions and methods for their use are provided in more detail below.

In yet another aspect, the present invention provides a method for modulating the action of an LXR receptor, preferably LXRα, in a cell. According to this method, the cell is contacted with a sufficient concentration of a composition containing a compound of formula I or II for either an agonistic or antagonistic effect to be detected. In preferred embodiments, the composition contains an amount of the compound which has been determined to provide a desired therapeutic or prophylactic effect for a given LXR-mediated condition.

In still another aspect, the present invention provides methods for the treatment of pathology such as obesity, diabetes, hypercholesterolemia, atherosclerosis, and hyperlipoproteinemia using pharmaceutical compositions containing compounds of the foregoing description of the general formulas I and II. Briefly, this aspect of the invention involves administering to a patient an effective formulation of one or more of the subject compositions. In other embodiments, the compound of formula I or II can be administered in combination with other anti-hypercholesterolemic agents (e.g., a bile acid sequestrant, nicotinic acid, fibric acid derivatives or HMG CoA reductase inhibitors), or in combination with other agents that affect cholesterol or lipid metabolism.

Preparation of the Compounds

Several methods for preparing the compounds of the present invention are illustrated in the following schemes and examples. Starting materials are made by known procedures or as illustrated. One of skill in the art will understand that similar methods can be used for the synthesis of the compounds.

As shown in Scheme 1, compounds of the present invention can be prepared beginning with commercially available 2,2,2,2′-tetrafluoroacetophenone (1). Treatment of 1 with an N-substituted arylsulfonamide (2) in the presence of a base such as potassium carbonate, cesium carbonate or sodium hydride in a suitable solvent such as DMF or DMSO provides adduct 3. Treatment of 3 with an appropriate organometallic species (4) provides compound 5.

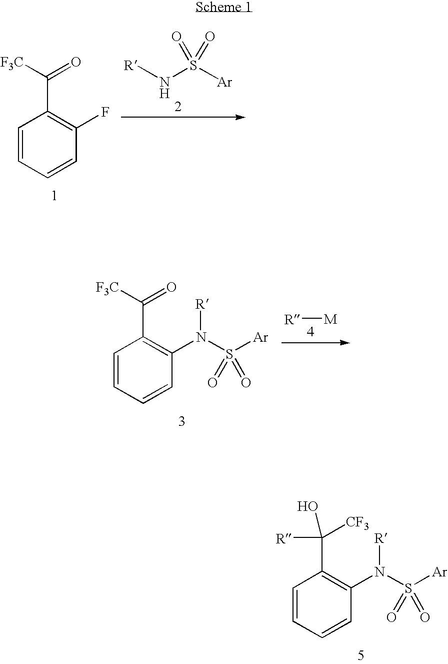

Another synthesis of the intermediate fluoroketone 3 is shown in Scheme 2. A 2-haloaniline (6) is sulfonylated with, for example, an appropriate sulfonyl halide, and subsequently alkylated with an appropriate alkylhalide in the presence of a base such as potassium carbonate, cesium carbonate or sodium hydride in a suitable solvent such as DMF or DMSO to provide compound 7. Halo-substituted arylsulfonamide 7 can be converted into fluoroketone 3 upon treatment with n-butyllithium or t-butyllithium followed by addition of, for example, ethyl trifluoroacetate (8).

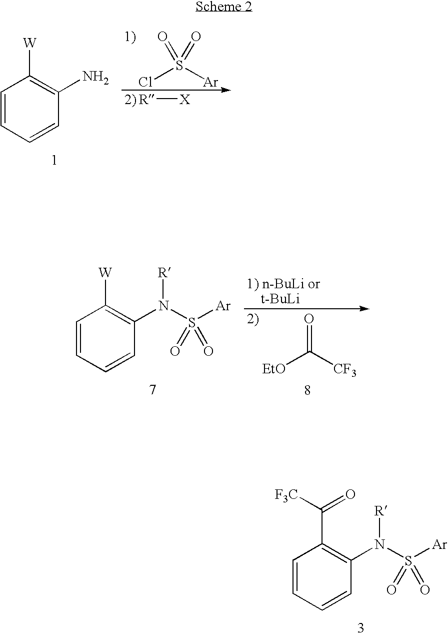

Scheme 3 illustrates the preparation of exemplary organometallic species 4. Briefly, an alkyne (9) can be lithiated with, for example, n-butyllithium in THF, or metalated with isopropylmagnesium bromide in THF.

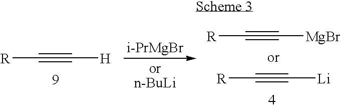

The preparation of alkynes 9 is illustrated in Scheme 4. An alkyl or aryl or heteroaryl halide (10) can be coupled to 2-methyl-3-butyn-2-ol (11) according to the procedure described in Bleicher et al. (1995) Synlett 1115-1116. The resulting alcohol 12, can be converted to alkyne 9 using a base such as sodium hydride in a suitable solvent such as toluene according to the procedure described in Havens et al. (1985) J. Org. Chem. 50:1763.

Alternatively an alkyl or aryl or heteroaryl halide can be coupled to ethynyltrimethylsilane (13) via a palladium mediated coupling reaction to afford 14 (see, e.g., R. C. Larock; Comprehensive Organic Transformations, 2^{nd} ed., John Wiley & Sons: New York, 1999; pp. 596-599). Subsequent treatment of 14 with, for example, potassium carbonate in anhydrous methanol, gives alkyne 9.

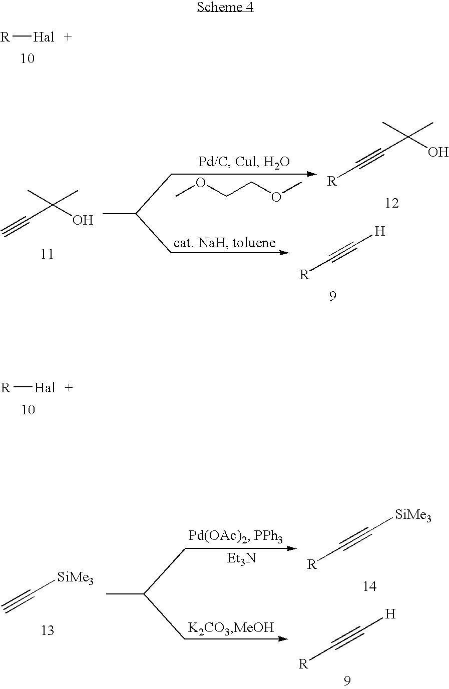

Other compounds of this invention can be prepared as shown in Scheme 5. A 3-haloaniline (15) is sulfonylated with, for example, an appropriate sulfonyl halide, and subsequently alkylated with an appropriate alkylhalide in the presence of a base such as potassium carbonate, cesium carbonate or sodium hydride in a suitable solvent such as DMF or DMSO to provide 16. Halo-substituted arylsulfonamide 16 can be converted into fluoroketone 17 by treatment with n-butyllithium or t-butyllithium followed by addition of, for example, ethyl trifluoroacetate (8). Treatment of 17 with organometallic species 4 provides 18.

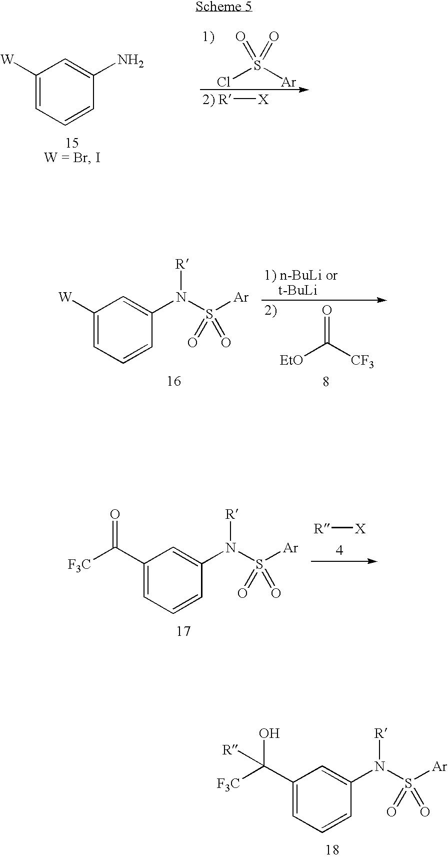

An alternative preparation of the target compounds is shown in Scheme 6: 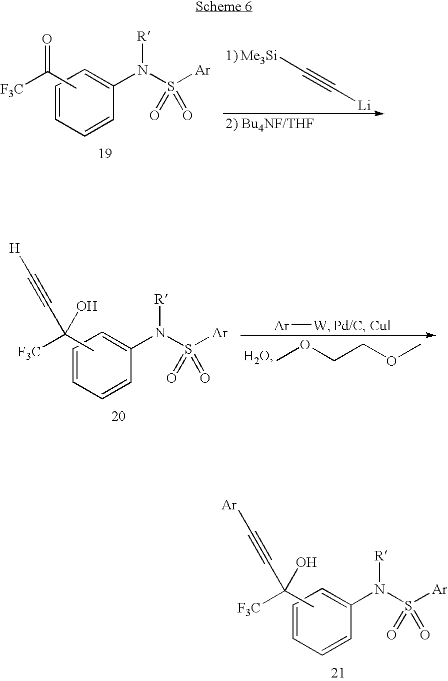
Treatment of 19 with trimethylsilyl-ethynyl lithium followed by tetrabutyl ammonium fluoride in THF affords ethynyl derivative 20. Reaction of 20 with an alkyl, aryl or heteroaryl halide using the procedure described in Bleicher et al. (1995) Synlett 1115-1116, or a similar palladium mediated coupling reaction (see, e.g., R. C. Larock; Comprehensive Organic Transformations, 2^{nd} ed., John Wiley & Sons: New York, 1999; pp. 596-599) affords 21.

As shown in Scheme 7, alcohol 21 can be alkylated in the presence of a base such as sodium hydride in a suitable solvent such as THF or DMF to give ether 22, or deoxygenated using, e.g., triethylsilane and BF₃.OEt₂, to give 23. 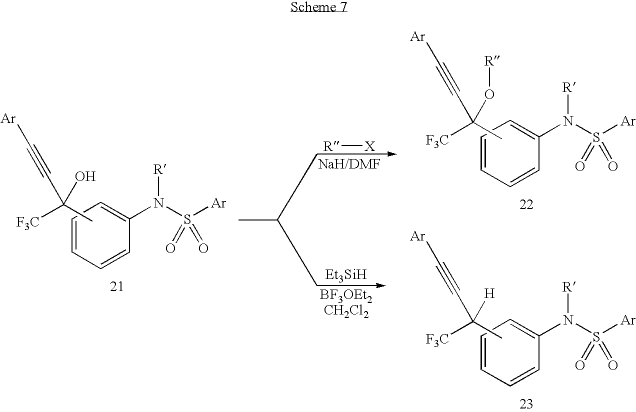
Analysis of the Compounds

Representative compounds and compositions were demonstrated to have pharmacological activity in in vitro and in vivo assays, e.g., they are capable of specifically modulating a cellular physiology to reduce an associated pathology or provide or enhance a prophylaxis.

Certain preferred compounds and compositions are capable of specifically regulating LXR. Compounds may be evaluated in vitro for their ability to activate LXR receptor function using biochemical assays (see U.S. Pat. No. 6,555,326 and U.S. patent application Ser. No. 09/163,713 (filed Sep. 30, 1998)), or in cell-based assays such as that described in Lehmann et al. (1997) J. Biol. Chem. 272(6):3137-3140). Alternatively, the compounds and compositions can be evaluated for their ability to increase or decrease gene expression modulated by LXR, using western-blot analysis. Established animal models to evaluate hypocholesterolemic effects of the compounds are also known in the art. For example, compounds disclosed herein can lower cholesterol levels in hamsters fed a high-cholesterol diet, using a protocol similar to that described in Spady et al. (1988) J. Clin. Invest. 81:300), Evans et al. (1994) J. Lipid Res. 35:1634, and Lin et al. (1995) J. Med. Chem. 38:277). Still further, LXRα animal models (e.g., LXRα (+/−) and (−/−) mice) can be used for evaluation of the present compounds and compositions (see, for example, Peet et al. (1998) Cell 93:693-704).

Accordingly, as used herein, the term “LXR-modulating amount” refers to that amount of a compound that is needed to produce a desired effect in any one of the cell-based assays, biochemical assays or animal models described above. Typically, an LXR-modulating amount of a compound will be at least that amount which exhibits an EC₅₀ in a reporter-gene cell-based assay (relative to an untreated control).

Formulation and Administration of Compounds and Pharmaceutical Compositions

The invention provides methods of using the subject compounds and compositions to treat disease or provide medicinal prophylaxis, to activate LXR receptor function in a cell, to reduce blood cholesterol concentration in a host, to slow down and/or reduce the abnormal cellular proliferation including the growth of tumors, etc. These methods generally involve contacting the cell or cells with or administering to a host an effective amount of the subject compounds or pharmaceutically acceptable compositions.

The compositions and compounds of the invention and the pharmaceutically acceptable salts or prodrugs thereof can be administered in any effective way such as via oral, parenteral or topical routes. Generally, the compounds are administered in dosages ranging from about 2 mg up to about 2,000 mg per day, although variations will necessarily occur depending on the disease target, the patient, and the route of administration. Preferred dosages are administered orally in the range of about 0.05 mg/kg to about 20 mg/kg, more preferably in the range of about 0.05 mg/kg to about 2 mg/kg, most preferably in the range of about 0.05 mg/kg to about 0.2 mg per kg of body weight per day.

In one embodiment, the invention provides the subject compounds combined with a pharmaceutically acceptable excipient such as sterile saline or other medium, water, gelatin, an oil, etc. to form pharmaceutically acceptable compositions. The compositions and/or compounds may be administered alone or in combination with any convenient carrier, diluent, etc. and such administration may be provided in single or multiple dosages. Useful carriers include solid, semi-solid or liquid media including water and non-toxic organic solvents.

In another embodiment, the invention provides the subject compounds in the form of a prodrug, which can be metabolically converted to the subject compound by the recipient host. A wide variety of prodrug formulations are known in the art.

The compositions may be provided in any convenient form including tablets, capsules, lozenges, troches, hard candies, powders, sprays, creams, suppositories, etc. As such the compositions, in pharmaceutically acceptable dosage units or in bulk, may be incorporated into a wide variety of containers. For example, dosage units may be included in a variety of containers including capsules, pills, etc.

The compositions may be advantageously combined and/or used in combination with other hypocholesterolemic therapeutic or prophylactic agents, different from the subject compounds. In many instances, administration in conjunction with the subject compositions enhances the efficacy of such agents. Exemplary hypocholesterolemic and/or hypolipemic agents include: bile acid sequestrants such as quaternary amines (e.g. cholestyramine and colestipol); nicotinic acid and its derivatives; HMG-CoA reductase inhibitors such as mevastatin, pravastatin, and simvastatin; gemfibrozil and other fibric acids, such as clofibrate, fenofibrate, benzafibrate and cipofibrate; probucol; raloxifene and its derivatives; and mixtures thereof.

The compounds and compositions also find use in a variety of in vitro and in vivo assays, including diagnostic assays. For example, various allotypic LDL receptor gene expression processes may be distinguished in sensitivity assays with the subject compounds and compositions, or panels thereof. In certain assays and in in vivo distribution studies, it is desirable to use labeled versions of the subject compounds and compositions, e.g. radioligand displacement assays. Accordingly, the invention provides the subject compounds and compositions comprising a detectable label, which may be spectroscopic (e.g. fluorescent), radioactive, etc.

The following examples are offered by way of illustration and not by way of limitation.

### EXAMPLES

¹H-NMR spectra were recorded on a Varian Gemini 400 MHz NMR spectrometer. Significant peaks are tabulated and typically include: number of protons, multiplicity (s, singlet; d, doublet; t, triplet; q, quartet; m, multiplet; br s, broad singlet) and coupling constant(s) in Hertz. Electron Ionization (EI) mass spectra were recorded on a Hewlett Packard 5989A mass spectrometer. Mass spectrometry results are reported as the ratio of mass over charge, followed by the relative abundance of each ion (in parentheses). Starting materials in the synthesis examples below are either available from commercial sources such as Aldrich Chemical Co., Milwaukee, Wis., USA, or via literature procedures. Abbreviations used in the examples below have their accepted meanings in the chemical literature. For example, THF (tetrahydrofuran), Et₂O (diethyl ether), MeOH (methanol), CH₂Cl₂ (methylene chloride), LDA (lithium diisopropylamide), MeCN (acetonitrile), DMAP (4-dimethyaminopyridine) and DMF (dimethylformamide).

### Example 1

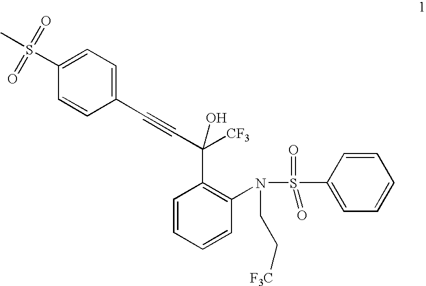

### N-{2-[1-Hydroxy-3-(4-methanesulfonylphenyl)-1-trifluoromethyl-prop-2-ynyl]-phenyl}-N-(3,3,3-trifluoropropyl)-benzenesulfonamide (1)

Step A. 1-Ethynyl-4-methanesulfonyl-benzene. 2-Methyl-3-butyn-2-ol was coupled to 1-bromo-4-methanesulfonyl-benzene according to the procedure described in Bleicher et al. (1995) Synlett 1115-1116. The product was converted to 1-ethynyl-4-methanesulfonyl-benzene according to the procedure described in Havens et al. (1985) J. Org. Chem. 50:1763-1765. ¹H NMR (CDCl₃) δ 3.06 (s, 3H), 3.29 (s, 1H), 7.67 (d, J=8.1 Hz, 2H), 7.91 (d, J=8.1 Hz, 2H).

Step B. N-(3,3,3-Trifluoropropyl)-benzenesulfonamide. To a solution of 1.00 g (6.7 mmol) of 3,3,3-trifluoropropylamine.HCl in 20 mL of dichloromethane at 0° C. was added 1.9 mL (13.3 mmol) of triethylamine and 431 μL (3.3 mmol) of benzenesulfonyl chloride sequentially. The mixture was allowed to gradually warm up to room temperature overnight (20 h) and diluted with dichloromethane. The resultant mixture was washed with saturated aqueous ammonium chloride (2×) and brine, dried over Na₂SO₄, filtered, and the filtrate was concentrated to give the title compound. ¹H-NMR (CDCl₃) δ 2.30-2.43 (m, 2H), 3.22 (q, J=6.7 Hz, 2H), 5.01 (br s, 1H), 7.48-7.65 (m, 3H), 7.82-7.94 (m, 2H). Mass Spectrum (ESI) m/e=254.1 (M+1).

Step C. N-(2-Trifluoroacetyl-phenyl)-N-(3,3,3-trifluoropropyl) benzenesulfonamide. To a suspension of 76 mg (1.90 mmol) of NaH (60% dispersion in mineral oil) in 7 mL of DMF at 0° C. was added a solution of 400 mg (1.58 mmol) of N-(3,3,3-trifluoropropyl)-benzenesulfonamide in 4 mL of DMF. The mixture was warmed to room temperature and stirred for 1 h. A solution of 328 mg (1.71 mmol) of 2,2,2,2′-tetrafluoroacetophenone in 3 mL of DMF was added and the resultant mixture was stirred at rt. After 23 h, the reaction mixture was concentrated, and the residue was dissolved in EtOAc and washed with saturated aqueous sodium bicarbonate (2×) and brine. The organic layer was dried over Na₂SO₄, filtered, and the filtrate was concentrated. The residue was purified by chromatography on silica gel (hexanes:EtOAc, 17:3) to give the title compound.

Step D. N-{2-[1-Hydroxy-3-(4-methanesulfonylphenyl)-1-trifluoromethyl-prop-2 ynyl]-phenyl}-N-(3,3,3-trifluoropropyl)-benzenesulfonamide. To a solution of 22 mg (0.12 mmol) of 1-ethynyl-4-methanesulfonyl-benzene (Example 1, Step A) in 4 mL of THF at −78° C. was added dropwise 47 μL (0.12 mmol) of n-BuLi (2.5 M solution in hexanes). After 40 min at −78° C., a solution of 43.5 mg (0.10 mmol) of N-(2-trifluoroacetyl-phenyl)-N-(3,3,3-trifluoropropyl)-benzenesulfonamide (Example 1, Step C) in 3 mL of THF was added and the resultant mixture was stirred at −78° C. for 2.5 h. The reaction mixture was quenched with saturated aqueous ammonium chloride and extracted with ethyl acetate (3×). The organic layers were dried over Na₂SO₄, filtered, and the filtrate was concentrated. The residue was purified by reverse phase preparatory HPLC (acetonitrile:water, 0.1% TFA) to give the title compound. ¹H-NMR (CDCl₃, mixture of rotamers) δ 2.28-2.45 (m, 1H, minor), 2.49-2.71 (m, 1H, major), 2.49-2.71 (m, 1H, minor), 2.75-2.91 (m, 1H, major), 3.06 (s, 3H, minor), 3.07 (s, 3H, major), 3.42-3.57 (m, 1H), 3.89-4.06 (m, 1H), 5.35 (s, 1H, minor), 5.81 (s, 1H, major), 6.40 (d, J=8.0 Hz, 1H, major), 6.57 (d, J=8.0 Hz, 1H, minor), 7.25 (dt, J=8.0 Hz, 1.5 Hz, 1H, major), 7.33 (dt, J=8.0 Hz, 1.5 Hz, 1H, minor), 7.41-7.78 (m, 8H), 7.93 (dd, J=8.5 Hz, 5.4 Hz, 2H), 7.99 (t, J=7.2 Hz, 1H). Mass Spectrum (ESI) m/e=606.1 (M+1), 623.0 (M+18), 628.0 (M+23).

### Example 2

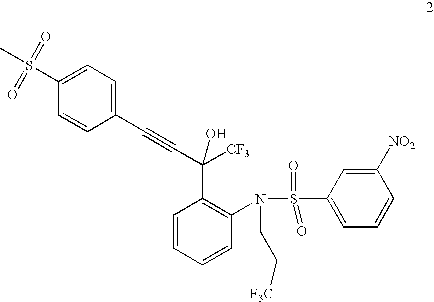

### 3-Nitro-N-{2-[1-hydroxy-3-(4-methanesulfonylphenyl)-1-trifluoromethyl-prop-2-ynyl]-phenyl}-N-(3,3,3-trifluoropropyl)-benzenesulfonamide (2)

Step A. 3-Nitro-N-(3,3,3-trifluoropropyl)-benzenesulfonamide. The title compound was prepared as described in Example 1, Step B. ¹H-NMR (CDCl₃) δ 2.35-2.48 (m, 2H), 3.32 (t, J=6.6 Hz, 2H), 5.23 (br s, 1H), 7.79 (t, J=8.1 Hz, 1H), 8.21 (dt, J=7.8 Hz, 1.1 Hz, 1H), 8.46 (dq, J=8.2 Hz, 1.0 Hz, 1H), 8.72 (t, J=1.3 Hz, 1H). Mass Spectrum (ESI) m/e=317.3 (M+19).

Step B. 3-Nitro-N-{2-[1-hydroxy-3-(4-methanesulfonylphenyl)-1-trifluoromethyl-prop-2-ynyl]-phenyl}-N-(3,3,3-trifluoropropyl)-benzenesulfonamide. The title compound was prepared as described in Example 1, Steps C and D. ¹H-NMR (CDCl₃, mixture of rotamers) δ 2.32-2.87 (m, 2H), 3.07 (s, 3H), 3.55-3.65 (m, 1H, minor), 3.67-3.77 (m, 1H, major), 3.91-4.05 (m, 1H), 4.48 (s, 1H, minor), 4.96 (s, 1H, major), 6.52 (dd, J=8.0 Hz, 1.2 Hz, 1H, major), 6.62 (dd, J=8.0 Hz, 1.2 Hz, 1H, minor), 7.25-7.41 (m, 1H), 7.47-7.59 (m, 1H), 7.70-7.82 (m, 3H), 7.91-8.08 (m, 4H), 8.39 (t, J=7.0 Hz, 1H, major), 8.47-8.58 (m, 1H), 8.54 (s, 1H, minor). Mass Spectrum (ESI) m/e=650.0 (M+1), 673.1 (M+23).

### Example 3

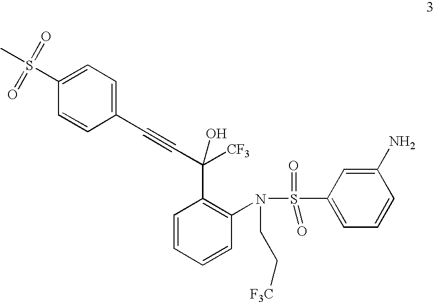

3-Amino-N-{2-[1-hydroxy-3-(4-methanesulfonylphenyl)-1-trifluoromethyl-prop-2-ynyl]-phenyl}-N-(3,3,3-trifluoropropyl)-benzenesulfonamide (3). To a solution of 255 mg (0.39 mmol) of the 3-nitro-N-{2-[1-hydroxy-3-(4-methanesulfonylphenyl)-1-trifluoromethyl-prop-2-ynyl]-phenyl}-N-(3,3,3-trifluoropropyl)-benzenesulfonamide (Example 9) in 10 mL of EtOAc and 10 mL of EtOH was added 364 mg (1.58 mmol) of tin(II) chloride dihydrate. The mixture was heated to reflux for 2 h. The reaction mixture was cooled to room temperature, quenched with 1 N HCl and extracted with EtOAc (3×). The organic layers were dried over Na₂SO₄, filtered, and the filtrate was concentrated. The residue was purified by chromatography on silica gel (hexanes:EtOAc, 11:9 grading to hexanes:EtOAc, 1:1) to give the title compound. ¹H-NMR (CDCl₃, mixture of rotamers) δ 2.25-2.91 (m, 2H), 3.05 (s, 3H, minor), 3.06 (s, 3H, major), 3.45-3.56 (m, 1H), 3.93 (dt, J=12.8 Hz, 4.9 Hz, 1H), 4.02 (ddd, J=16.8 Hz, 14.1 Hz, 5.2 Hz, 1H), 5.46 (br s, 1H, minor), 5.89 (s, 1H, major), 6.54 (dd, J=8.0 Hz, 1.2 Hz, 1H, minor), 6.70 (dd, J=8.0 Hz, 1.2 Hz, 1H, major), 6.84-6.88 (m, 1H, minor), 6.89-6.98 (m, 2H), 7.06 (d, J=7.9 Hz, 1H, major), 7.24-7.39 (m, 2H), 7.41-7.53 (m, 1H), 7.74 (dd, J=8.1 Hz, 6.5 Hz, 2H), 7.91-8.02 (m, 3H). Mass Spectrum (ESI) m/e=621.0 (M+1), 643.0 (M+23).

### Example 7

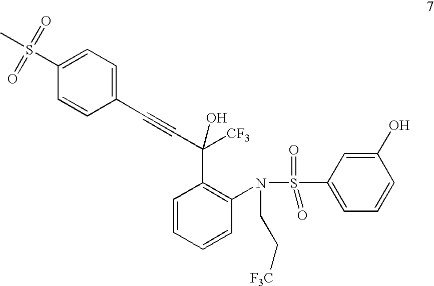

3-Hydroxy-N-{2-[1-hydroxy-3-(4-methanesulfonylphenyl)-1-trifluoromethyl-prop-2-ynyl]-phenyl}-N-(3,3,3-trifluoropropyl)-benzenesulfonamide (7). To a suspension of 53 mg (0.09 mmol) of 3-amino-N-{2-[1-hydroxy-3-(4-methanesulfonylphenyl)-1-trifluoromethyl-prop-2-ynyl]-phenyl}-N-(3,3,3-trifluoropropyl)-benzenesulfonamide (Example 3) in 2.6 mL of water and 0.4 mL of conc. HCl at 0° C. was added dropwise a solution of 6.7 mg (0.09 mmol) of sodium nitrite in 0.4 mL of water. After 1 h at 0° C., the mixture was heated to reflux for 2.5 h. The reaction mixture was cooled to room temperature and extracted with CH₂Cl₂. The organic layer was dried over Na₂SO₄, filtered, and the filtrate was concentrated. The residue was purified by chromatography on silica gel (hexanes:EtOAc, 13:7) to give the title compound. ¹H-NMR (CDCl₃, mixture of rotamers) δ 2.31-2.86 (m, 2H), 3.06 (s, 3H, major), 3.07 (s, 3H, minor), 3.46-3.59 (m, 1H), 3.84-3.93 (m, 1H, major), 3.97-4.06 (m, 1H, minor), 5.34 (br s, 1H, minor), 5.76 (s, 1H, major), 6.55 (dd, J=8.0 Hz, 1.1 Hz, 1H, major), 6.63 (dd, J=7.9 Hz, 1.2 Hz, 1H, minor), 7.08 (dt, J=5.5 Hz, 2.1 Hz, 1H), 7.11-7.19 (m, 1H), 7.26-7.47(m, 4H), 7.70-7.79 (m, 2H), 7.89-7.98 (m, 3H). Mass Spectrum (ESI) m/e=622.0 (M+1), 639.1 (M+18), 644.0 (M+23).

### Example 8

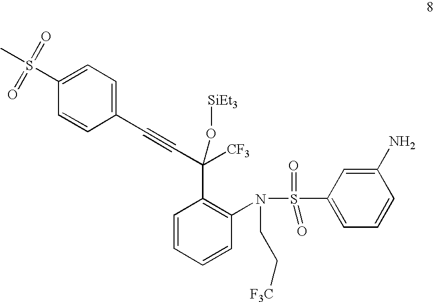

3-Amino-N-{2-[3-(4-methanesulfonylphenyl)-1-(triethylsilanyloxy)-1-trifluoromethyl-prop-2-ynyl]-phenyl}-N-(3,3,3-trifluoropropyl)-benzenesulfonamide (8). To a solution of 33 mg (0.05 mmol) of 3-amino-N-{2-[1-hydroxy-3-(4-methanesulfonylphenyl)-1-trifluoromethyl-prop-2-ynyl]-phenyl}-N-(3,3,3-trifluoropropyl)-benzenesulfonamide (Example 3) and 36 mg (0.53 mmol) of imidazole in 3.5 mL DMF was added 45 μL (0.27 mmol) of chlorotriethylsilane. The mixture was stirred for 18 h. The reaction mixture was quenched with a mixture of water and brine and extracted with ethyl acetate (3×). The organic layers were dried over Na₂SO₄, filtered, and the filtrate was concentrated. The residue was purified by chromatography on silica gel (hexanes:EtOAc, 13:7) to give the title compound. ¹H-NMR (CDCl₃) δ 0.69-0.91 (m, 6H), 0.95 (t, J=7.8 Hz, 9H), 2.51-2.68 (m, 1H), 2.70-2.88 (m, 1H), 3.09 (s, 3H), 3.39-3.49 (m, 1H), 3.76-3.87 (m, 1H), 3.92 (s, 2H), 6.50 (dd, J=7.9 Hz, 1.2 Hz, 1H), 6.89-6.95 (m, 2H), 7.01 (d, J=8.0 Hz, 1H), 7.25-7.34 (m, 2H), 7.45 (dt, J=8.4 Hz, 1.3 Hz, 1H), 7.88 (d, J=8.6 Hz, 2H), 7.96 (d, J=8.6 Hz, 2H), 8.02 (d, J=8.1 Hz, 1H). Mass Spectrum (ESI) m/e=735.0 (M+1).

### Example 9

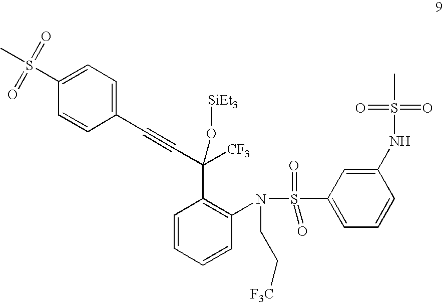

3-Methanesulfonylamino-N-{2-[3-(4-methanesulfonylphenyl)-1-(triethylsilanyloxy)-1-trifluoromethyl-prop-2-ynyl]-phenyl}-N-(3,3,3-trifluoropropyl)-benzenesulfonamide (9). To a solution of 9.5 mg (0.01 mmol) of 3-amino-N-{2-[3-(4-methanesulfonylphenyl)-1-(triethylsilanyloxy)-1-trifluoromethyl-prop-2-ynyl]-phenyl}-N-(3,3,3-trifluoropropyl)-benzenesulfonamide (Example 8) in 2 mL of dichloromethane was added 30 μL (0.26 mmol) of 2,6-lutidine and 10 μL (0.13 mmol) of methanesulfonyl chloride. The mixture was stirred for 5.5 h. The reaction mixture was quenched with 1 N HCl and extracted with ethyl acetate (3×). The organic layers were dried over Na₂SO₄, filtered, and the filtrate was concentrated. The residue was purified by chromatography on silica gel (hexanes:EtOAc, 9:11) to give the title compound. ¹H-NMR (CDCl₃) δ 0.71-0.90 (m, 6H), 0.95 (t, J=7.9 Hz, 9H), 2.51-2.84 (m, 2H), 3.08 (s, 3H), 3.10 (s, 3H), 3.44-3.56 (m, 1H), 3.71-3.82 (m, 1H), 6.42 (dd, J=7.9 Hz, 1.2 Hz, 1H), 6.73 (s, 1H), 7.25-7.34 (m, 1H), 7.40-7.60 (m, 5H), 7.87 (d, J=8.6 Hz, 2H), 7.97 (d, J=8.6 Hz, 2H), 8.04 (d, J=8.1 Hz, 1H). Mass Spectrum (ESI) m/e=835.0 (M+23).

### Example 10

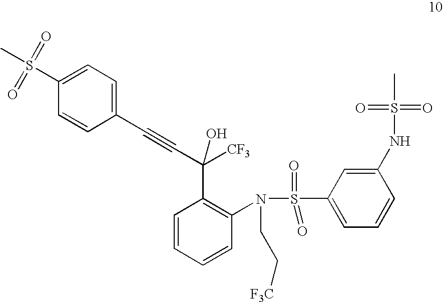

N-{2-[1-Hydroxy-3-(4-methanesulfonylphenyl)-1-trifluoromethyl-prop-2-ynyl]-phenyl}-3-methanesulfonylamino-N-(3,3,3-trifluoropropyl)-benzenesulfonamide (10). To a solution of 4.2 mg (0.005 mmol) of 3-methanesulfonylamino-N-{2-[3-(4-methanesulfonylphenyl)-1-(triethylsilanyloxy)-1-trifluoromethyl-prop-2-ynyl]-phenyl}-N-(3,3,3-trifluoropropyl)-benzenesulfonamide (Example 9) in 2.5 mL of THF was added 18 μL (0.02 mmol) of tetrabutylammonium fluoride (1.0 M solution in THF) dropwise. The mixture was stirred for 3 h. The reaction mixture was quenched with brine and extracted with ethyl acetate (3×). The organic layers were dried over Na₂SO₄, filtered, and the filtrate was concentrated. The residue was purified by chromatography on silica gel (hexanes:EtOAc, 1:1) to give the title compound. ¹H-NMR (CDCl₃, mixture of rotamers) δ 2.50-2.87 (m, 2H), 3.00 (br s, 3H, minor), 3.02 (s, 3H, major), 3.06 (s, 3H, minor), 3.08 (s, 3H, major), 3.51-3.72 (m, 1H), 3.86-4.02 (m, 1H), 6.68 (d, J=7.5 Hz, 1H, major), 6.74 (d, J=8.1 Hz, 1H, minor), 7.26-7.38 (m, 1H), 7.39-7.55 (m, 5H), 7.72 (dd, J=8.2 Hz, 3.5 Hz, 2H), 7.88-7.99 (m, 3H). Mass Spectrum (ESI) m/e=699.0 (M+1), 716.0 (M+18), 721.0 (M+23).

### Example 11

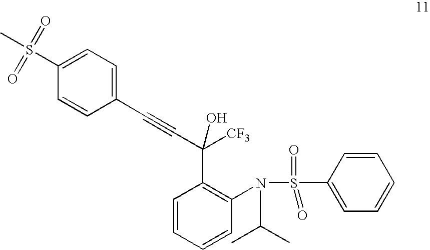

N-{2-[1-Hydroxy-3-(4-methanesulfonylphenyl)-1-trifluoromethyl-prop-2ynyl]-phenyl-N-isopropyl-benzenesulfonamide (11).

Step A. N-(2-Bromophenyl)-benzenesulfonamide. To a solution of 9.4 mL (73.7 mmol) of benzenesulfonyl chloride in 70 mL of dichloromethane at 0° C. was added 11.0 mL (136.0 mmol) of pyridine and 10.0 mL (98%, 86.6 mmol) of 2-bromoaniline sequentially. The mixture was allowed to gradually warm up to room temperature overnight (19 h) and diluted with dichloromethane. The resultant mixture was washed with saturated aqueous ammonium chloride, 1 M citric acid solution (2×), saturated aqueous sodium bicarbonate and brine, dried over Na₂SO₄, filtered, and the filtrate was concentrated to give the title compound. ¹H-NMR (CDCl₃) δ 4.08 (br s, 1H), 6.93-7.01 (m, 1H), 7.25-7.31 (m, 1H), 7.38-7.45 (m, 3H), 7.51-7.58 (m, 1H), 7.68 (dd, J=8.2 Hz, 1.5 Hz, 1H), 7.76 (dd, J=8.3 Hz, 1.25 Hz, 2H). Mass Spectrum (ESI) m/e=312.0 (M+1), 329.0 (M+18).

Step B. N-(2-Bromophenyl)-N-isopropyl-benzenesulfonamide. To a suspension of 1.15 g (28.8 mmol) of NaH (60% dispersion in oil) in 20 mL of DMF was added a solution of 7.50 g (24.0 mmol) of N-(2-bromophenyl)-benzenesulfonamide in 10 mL of DMF. The mixture was stirred for 1.25 h and 2.90 mL (28.8 mmol) of 2-iodopropane was added. The resultant mixture was stirred for 18 h. The reaction mixture was quenched with saturated aqueous ammonium chloride and extracted with EtOAc. The organic layer was washed with saturated aqueous sodium bicarbonate and brine, dried over Na₂SO₄, filtered, and the filtrate was concentrated. The residue was purified by chromatography on silica gel (hexanes:ethyl ether, 17:3) to give the title compound. ¹H-NMR (CDCl₃) δ 1.05 (d, J=6.7 Hz, 3H), 1.18 (d, J=6.7 Hz, 3H), 4.47 (quintet, J=6.7 Hz, 1H), 7.11 (d, J=7.7 Hz, 1H), 7.19-7.31 (m, 2H), 7.48 (t, J=7.7 Hz, 2H), 7.57 (d, J=Hz, 1H), 7.63-7.71 (m, 1H), 7.82 (d, J=7.8 Hz, 2H). Mass Spectrum (ESI) m/e=354.0 (M+1), 376.0 (M+23).

Step C. N-Isopropyl-N-(2-trifluoroacetyl-phenyl)-benzenesulfonamide. To a solution of 1.0 g (2.8 mmol) of N-(2-bromophenyl)-N-isopropyl-benzenesulfonamide in 30 mL of THF at −78° C. was added 1.18 mL (3.0 mmol) of n-butyllithium (2.5 M solution in hexanes) dropwise. The mixture was stirred for 15 min at −78° C. and 370 μL (3.1 mmol) of ethyl trifluoroacetate was added in a single portion. The resultant mixture was stirred at −78° C. for 35 min, warmed to 0° C. and stirred for an additional 5 min. The reaction mixture was quenched with saturated aqueous ammonium chloride and extracted with EtOAc. The organic layer was washed with brine, dried over Na₂SO₄, filtered, and the filtrate was concentrated. The residue was purified by chromatography on silica gel (hexanes:EtOAc, 9:1) to give the title compound. ¹H-NMR (CDCl₃) δ 1.05 (d, J=6.7 Hz, 6H), 4.50 (quintet, J=6.7 Hz, 1H), 7.03-7.07 (m, 2H), 7.33-7.42 (m, 4H), 7.58-7.69 (m, 3H). Mass Spectrum (ESI) m/e=372.1 (M+1), 389.0 (M+18).

Step D. N-{2-[1-Hydroxy-3-(4-methanesulfonylphenyl)-1-trifluoromethyl-prop-2-ynyl]-phenyl-N-isopropyl-benzenesulfonamide. The title compound was prepared as described in Example 1, Step D. ¹H-NMR (CDCl₃) δ 1.13 (d, J=6.7 Hz, 3H), 1.17 (d, J=6.7 Hz, 3H), 3.05 (s, 3H), 4.62 (quintet, J=6.6 Hz, 1H), 7.14 (dd, J=7.9 Hz, 1.4 Hz, 1H), 7.28 (s, 1H), 7.29-7.38 (m, 4H), 7.44 (t, J=7.2 Hz, 1H), 7.59 (dt, J=7.7 Hz, 1.4 Hz, 1H), 7.72 (d, J=8.4 Hz, 2H), 7.90-7.95 (m, 3H), 7.99 (d, J=8.0 Hz, 2H). Mass Spectrum (ESI) m/e=552.1 (M+1), 574.0 (M+23).

The following compounds were prepared as described in Example 1. The required acetylenes, when not commercially available, were prepared as described in Example 1, Step A.

### Example 12

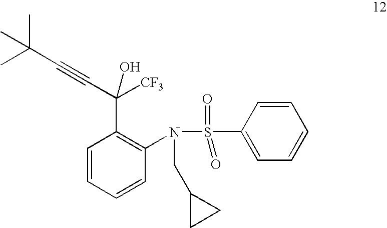

N-Cyclopropylmethyl-N-[2-(1-hydroxy-4,4-dimethyl-1-trifluoromethyl-pent-2-ynyl)-phenyl]-benzenesulfonamide (12). ¹H NMR (CDCl₃) δ 0.10-0.12 (m, 2H), 0.42-0.44 (m, 2H), 0.89-0.92 (m, 1H), 1.30 (s, 9H), 3.54 (dd, J=14.0 Hz, J=7.0 Hz, 1H), 3.61 (dd, J=14.0 Hz, J=7.0 Hz, 1H), 6.97 (s, 1H), 7.21-7.23 (m, 3H), 7.29-7.31 (m, 2H), 7.35-7.39 (m, 1H), 7.52-7.56 (m, 1H), 7.78-7.80 (m, 1H), 8.02-8.04 (m, 1H). Mass Spectrum (ESI) m/e=466 (M+1).

### Example 13

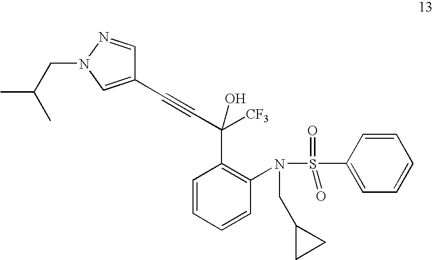

N-Cyclopropylmethyl-N-{2-[1-hydroxy-3-(1-isobutyl-1H pyrazol-3-yl)-1-trifluoromethyl-prop-2-ynyl]-phenyl}-benzenesulfonamide (13). ¹H NMR (CDCl₃) δ 0.10-0.14 (m, 2H), 0.44-0.46 (m, 2H), 0.88-0.92 (m, 1H), 0.929 (d, J=6.7 Hz, 6H), 2.21 (m, 1H), 3.55 (dd, J=14.0 Hz, J=6.8 Hz, 1H), 3.66 (dd, J=14.0 Hz, J=7.3 Hz, 1H), 3.91 (d, J=7.3 Hz, 2H), 7.13 (s, 1H), 7.23-7.34 (m, 5H), 7.39-7.43 (m, 1H), 7.57-7.59 (m, 1H), 7.61 (s, 1H), 7.68 (s, 1H), 7.80-7.83 (m, 1H), 8.08-8.10 (m, 1H). Mass Spectrum (ESI) m/e=532 (M+1).

### Example 15

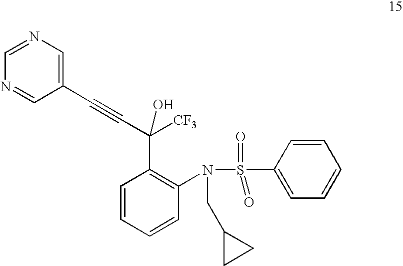

N-Cyclopropylmethyl-N-[2-(1-hydroxy-3-pyrimidin-5-yl-1-trifluoromethyl-prop-2-ynyl)-phenyl]-benzenesulfonamide (15). ¹H NMR (CDCl₃) δ 0.11-0.16 (m, 2H), 0.45-0.47 (m, 2H), 0.93-0.97 (m, 1H), 3.58 (dd, J=14.0 Hz, J=7.0 Hz, 1H), 3.66 (dd, J=14.0 Hz, J=7.3 Hz, 1H), 7.16 (s, 1H), 7.22-7.25 (m, 3H), 7.32-7.35 (m, 2H), 7.47-7.51 (m,1H), 7.59-7.63 (m, 1H), 7.90-7.92 (m, 1H), 7.96-7.98 (m, 1H), 8.87 (s, 2H), 9.20 (s, 1H). Mass Spectrum 488 (M+1).

The compounds listed in the following table were prepared according to the procedure described in Example 1.

TABLE 1

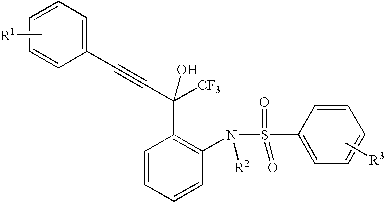

| Compound | R¹ | R² | R³ |
|---|---|---|---|
| 16 | 4-MeSO₂ | —CH₂CF₃ | H |
| 17 | 4-MeSO₂ | —CH₂CF₃ | 3-Cl |
| 18 | 4-MeSO₂ | —CH₂CH₂CF₃ | 2-Cl |
| 20 | 4-MeSO₂ | —CH₂CH₂CF₃ | 2,5-Cl₂ |
| 22 | 4-MeSO₂ | 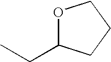 | 4-Cl |
| 23 | 4-MeSO₂ | 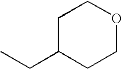 | H |

### Example 16

N-{2-[1-Hydroxy-3-(4-methanesulfonylphenyl)-1-trifluoromethyl-prop-2-ynyl]-phenyl}-N-(2,2,2-trifluoroethyl)-benzenesulfonamide (16). ¹H NMR (CDCl₃) δ 3.06 (s, 3H), 4.01 (m, 1H), 4.93 (m 1H), 6.56 (d, J=8.3 Hz, 1H), 7.17-7.95 (m 13H). Mass Spectrum (ESI) m/e=610 (M+H₃O⁺).

### Example 17

3-Chloro-N-{2-[1-Hydroxy-3-(4-methanesulfonylphenyl)-1-trifluoromethyl-prop-2-ynyl]-phenyl}-N-(2,2,2-trifluoroethyl)-benzenesulfonamide (17). ¹H NMR (CDCl₃) δ 3.07 (s, 3H), 3.96-4.23 (m, 1H), 4.72-4.94 (m, 1H), 6.66-6.75 (m, 1H), 7.25-7.99 (m 12H). Mass Spectrum (ESI) m/e=625 (M+1).

### Example 18

2-Chloro-N-{2-[1-hydroxy-3-(4-methanesulfonylphenyl)-1-trifluoromethyl-prop-2-ynyl]-phenyl}-N-(3,3,3-trifluoropropyl)-benzenesulfonamide (18). ¹H-NMR (CDCl₃, mixture of rotamers) δ 2.39-2.70 (m, 2H), 3.05 (s, 3H, minor), 3.06 (s, 3H, major), 3.91-4.00 (m, 1H, minor), 4.03-4.15 (m, 1H, major), 4.30-4.41 (1H, major), 4.30-4.41 (1H, minor), 5.35 (s, 1H, minor), 5.71 (s, 1H, major), 6.53 (dd, J=8.0 Hz, 1.3 Hz, 1H, major), 6.57 (dd, J=8.0 Hz, 1.3 Hz, 1H, minor), 7.13-7.76 (m, 8H), 7.90-7.99 (m, 3H). Mass Spectrum (ESI) m/e=640.0 (M+1).

### Example 20

2,5-Dichloro-N-{2-[1-hydroxy-3-(4-methanesulfonylphenyl)-1-trifluoromethyl-prop-2-ynyl]-phenyl}-N-(3,3,3-trifluoropropyl)-benzenesulfonamide (20). ¹H-NMR (CDCl₃, mixture of rotamers) δ 2.39-2.67 (m, 2H), 3.06 (s, 3H, minor), 3.07 (s, 3H, major), 3.43-3.63 (m, 1H), 4.00 (dt, J=12.4 Hz, 4.7 Hz, 1H, minor), 4.14 (dt, J=12.1 Hz, 4.8 Hz, 1H, major), 4.25-4.36 (m, 1H), 5.03 (s, 1H, minor), 5.34 (s, 1H, major), 6.59 (d, J=7.9 Hz, 1H, major), 6.63 (d, J=8.0 Hz, 1H, minor), 7.23 (t, J=7.6 Hz, 1H, major), 7.30 (t, J=7.7 Hz, 1H, minor), 7.45-7.56 (m, 3H), 7.42-7.56 (m, 3H), 7.67 (dd, J=15.0 Hz, 1.7 Hz, 1H), 7.70-7.79 (m, 2H), 7.90-8.03 (m, 3H). Mass Spectrum (ESI) m/e=674.0 (M+1), 691.0 (M+18), 1370.8 (2M+23).

### Example 22

4-Chloro-N-{2-[1-hydroxy-3-(4-methanesulfonylphenyl)-1-trifluoromethyl-prop-2-ynyl]-phenyl}-N-(tetrahydrofuran-2-ylmethyl)-benzenesulfonamide (22). ¹H-NMR (CDCl₃, mixture of rotamers/diastereomers) δ 1.78-1.94 (m, 2H), 3.05 (s, 3H, major), 3.06 (s, 3H, minor), 3.52-3.77 (m, 3H), 3.78-3.88 (m, 1H), 3.92-4.02 (m, 1H, minor), 4.15-4.24 (m, 1H, major), 6.37 (s, 1H, minor), 6.53 (s, 1H, major), 6.76 (dd, J=8.0 Hz, 1.1 Hz, 1H, major), 6.83 (d, J=6.9 Hz, 1H, minor), 7.24-7.34 (m, 1H), 7.39-7.48 (m, 3H), 7.63 (d, J=8.6 Hz, 2H), 7.68-7.77 (m, 3H), 7.84-7.95 (m, 3H). Mass Spectrum (ESI) m/e=628.0 (M+1), 650.0 (M+23).

### Example 23

N-{2-[1-Hydroxy-3-(4-methanesulfonylphenyl)-1-trifluoromethyl-prop-2-ynyl]-phenyl}-N-(tetrahydropyran-2-ylmethyl)-benzenesulfonamide (23). ¹H-NMR (CDCl₃) δ 1.71-1.83 (m, 2H), 2.02 (d, J=13.4 Hz, 1H), 3.05 (s, 3H), 3.22-3.35 (m, 3H), 3.73 (dd, J=13.6 Hz, 8.1 Hz, 1H), 3.90 (dd, J=11.5 Hz, 2.5 Hz, 1H), 3.97 (dd, J=11.5 Hz, 2.5 Hz, 1H), 6.30 (s, 1H), 6.56 (d, J=8.0 Hz, 1H), 7.19-7.25 (m, 1H), 7.35 (s, 1H), 7.39-7.75 (m, 5H), 7.72 (d, J=8.2 Hz, 2H), 7.93 (d, J=8.2 Hz, 3H). Mass Spectrum (ESI) m/e=608.0 (M+1), 630.1 (M+23).

The compounds in the following table were prepared according to the procedure described in Example 1.

TABLE 2

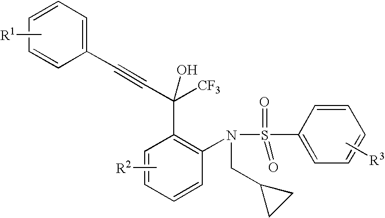

| Compound | R¹ | R² | R³ |
|---|---|---|---|
| 25 | H | H | H |
| 26 | 4-MeSO₂ | 4-Me | H |
| 29 | 4-MeSO₂ | 3-CF₃ | H |
| 30 | 3-MeSO₂ | 4-Cl | H |
| 31 | 4-MeSO₂ | 3-Cl | H |
| 33 | 4-MeSO₂ | H | 2-Cl |
| 34 | 4-MeSO₂ | H | 3-Cl |
| 35 | 4-NHAc | H | 2-Cl |
| 37 | 4-Et | H | H |
| 38 | 4-CF₃ | H | H |
| 39 | 3-Ph | H | H |
| 40 | 4-ⁱBuSO₂ | H | H |
| 41 | 4-MeO | H | H |
| 42 | 3-MeSO₂ | H | H |
| 43 | 4-MeSO₂ | H | H |
| 44 | 4-Pr(Me)NSO₂ | H | H |
| 45 | 4-MeN(H)CO | H | H |
| 47 | 4-Me₂NCH₂CH₂N(Me)CO | H | H |

### Example 25

N-Cyclopropylmethyl-N-[2-(1-hydroxy-3-phenyl-1-trifluoromethyl-prop-2-ynyl)-phenyl]-benzenesulfonamide (25). ¹H NMR (CDCl₃) δ 0.09 (m, 2H), 0.42 (m, 2H), 0.93 (m, 1H), 3.55 (dd, J=7.0 Hz, 13.9 Hz, 1H), 3.64 (dd, J=7.0 Hz, 13.9 Hz, 1H), 7.15 (s, 1H), 7.22-7.82 (m, 13H), 8.09 (d, J=8.1 Hz, 2H). Mass Spectrum (m/e)=486 (M+1).

### Example 26

N-Cyclopropylmethyl-N-{2-[1-hydroxy-3-(4-methanesulfonylphenyl)-1-trifluoromethyl-prop-2-ynyl]-4-methyl-phenyl}-benzenesulfonamide (26). ¹H NMR (CDCl₃) δ 0.10 (m, 2H), 0.43 (m, 2H), 0.92 (m, 1H), 2.32 (s, 3H), 3.05 (s, 3H), 3.52 (dd, J=7.2 Hz, 13.9 Hz, 1H), 3.60 (dd, J=7.2 Hz, 13.9 Hz, 1H), 7.06-7.98 (m, 13H). Mass Spectrum (m/e)=578 (M+1).

### Example 29

N-Cyclopropylmethyl-N-{2-[1-hydroxy-3-(4-methanesulfonylphenyl)-1-trifluoromethyl-prop-2-ynyl]-3-trifluoromethyl-phenyl}-benzenesulfonamide (29). ¹H NMR (DMSO) δ −0.26 to 0.02 (m, 2H), 0.26 (m, 2H), 0.71 (m, 1H), 3.32 (s, 3H), 3.41-3.97 (m, 2H), 6.81-8.28 (m, 13H). Mass Spectrum (ESI) m/e=745 (M+TFA).

### Example 30

N-Cyclopropylmethyl-N-{2-[1-hydroxy-3-(3-methanesulfonylphenyl)-1-trifluoromethyl-prop-2-ynyl]-4-chloro-phenyl}-benzenesulfonamide (30). ¹H NMR (CDCl₃, mixture of rotamers) δ −0.17-0.09 (m, 2H), 0.37-0.49 (m, 2H), 0.86-0.96 (m, 1H), 3.09 (s, 3H), 3.38-3.59 (m, 2H), 6.74 (d, J=8.6 Hz, 0.5H), 6.86 (d, J=8.6 Hz, 0.5H), 7.24-7.33 (m, 1H), 7.51-7.98 (m, 9H), 8.11-8.12 (m, 1H). Mass Spectrum (ESI) m/e=598 (M+1).

### Example 31

N-Cyclopropylmethyl-N-{2-[1-hydroxy-3-(4-methanesulfonylphenyl)-1-trifluoromethyl-prop-2-ynyl]-3-chloro-phenyl}-benzenesulfonamide (31). ¹H NMR (CDCl₃) δ −0.03-0.28 (m, 2H), 0.50-0.67 (m, 2H), 1.07 (m, 1H), 3.22 (s, 3H), 3.48-3.74 9 m, 2H), 6.18-8.10 (m, 13H). Mass Spectrum (ESI) m/e=598 (M+1).

### Example 33

2-Chloro-N-cyclopropylmethyl-N-{2-[1-hydroxy-3-(4-methanesulfonylphenyl)-1-trifluoromethyl-prop-2-ynyl]-phenyl}-benzenesulfonamide (33). ¹H NMR (CDCl₃) δ 0.01-0.15 (m, 2H), 0.45-0.59 (m, 2H), 1.15 (m, 1H), 3.22 (s, 3H), 3.72-4.35 (m, 2H), 6.92-7.02 (m, 1H), 7.30-8.11 (m 12H). Mass Spectrum (ESI) m/e=598 (M+1).

### Example 34

3-Chloro-N-cyclopropylmethyl-N-{2-[1-hydroxy-3-(4-methanesulfonylphenyl)-1-trifluoromethyl-prop-2-ynyl]-phenyl}-benzenesulfonamide (34). ¹H NMR (CDCl₃) δ 0.12 (m, 2H), 0.49 (m, 2H), 0.96 (m, 1H), 3.06 (s, 3H), 3.48 (dd, J=7.6 Hz, 14.1 Hz, 1H), 3.65 (dd, J=7.6 Hz, 14.1 Hz, 1H), 6.26 (br s, 1H), 6.82 (d, J=8.0 Hz, 1H), 7.22-7.67 (m, 7H), 7.72 (d, J=7.8 Hz, 2H), 7.93 (d, J=7.8 Hz, 2H). Mass Spectrum (ESI) m/e=598 (M+1).

### Example 35

N-[4-(3-{2-[(2-Chlorobenzenesulfonyl)-cyclopropylmethylamino]-phenyl-4,4,4-trifluoro-3-hydroxy-but-1-ynyl)-phenyl]-acetamide (35). ¹H NMR (CDCl₃) δ −0.15-1.04 (m, 4H), 1.25 (m, 1H), 2.18 (s, 3H), 3.53-4.22 (m, 2H), 5.94 (s, 1H), 6.79-6.90 (m, 1H), 7.11-7.92 (m, 12H). Mass Spectrum (ESI) m/e=577 (M+1).

### Example 37

N-Cyclopropylmethyl-N-{2-[3-(4-ethylphenyl)-1-hydroxy-1-trifluoromethyl-prop-2-ynyl]-phenyl}-benzenesulfonamide (37). ¹H NMR (CDCl₃) δ 0.07-0.1 (m, 2H), 0.43-0.46 (m, 2H), 0.93-0.96 (m, 1H), 1.25 (t, J=7.6 Hz, 3H), 2.68 (q, J=7.6 Hz, 2H), 3.56 (dd, J=14.0 Hz, J=6.9 Hz, 1H), 3.66 (dd, J=14.0 Hz, J=7.1 Hz, 1H) 7.15 (s, 1H), 7.19-7.65 (m, 9H), 7.82 (m, 1H), 8.13 (d, J=8.1 Hz, 1H). Mass Spectrum (ESI) m/e=514 (M+1).

### Example 38

N-Cyclopropylmethyl-N-{2-[1-hydroxy-1-trifluoromethyl-3-(4-trifluoromethylphenyl)-prop-2-ynyl]-phenyl}-benzenesulfonamide (38). ¹H NMR (CDCl₃) δ 0.08-0.14 (m, 2H), 0.42-0.46 (m, 2H), 0.91-0.97 (m, 1H), 3.56 (dd, J=13.9 Hz, J=7.0 Hz, 1H), 3.65 (dd, J=13.9 Hz, J=7.2 Hz, 1H), 7.16 (s, 1H), 7.22 (m, 3H), 7.31-7.34 (m, 2H), 7.43-7.47 (m, 1H), 7.58-7.67 (m, 5H), 7.85-7.88 (m, 1H), 8.02-8.04 (m, 1H). Mass Spectrum (ESI) m/e=554 (M+1).

### Example 39

N-{2-[3-Biphen-3-yl-1-hydroxy-1-trifluoromethyl-prop-2-ynyl]-phenyl}-N-cyclopropylmethyl benzenesulfonamide (39). ¹H NMR (CDCl₃) δ 0.09-0.14 (m, 2H), 0.42-0.46 (m, 2H), 0.91-0.97 (m, 1H), 3.57 (dd, J=14.0 Hz, J=7.0 Hz, 1H), 3.67 (dd, J=14.0 Hz, J=7.3 Hz, 1H), 7.19(s, 1H), 7.24-7.62 (m, 15H), 7.79 (s, 1H), 7.84 (d, J=8.1 Hz, 1H), 8.1 (d, J=8.1 Hz, 1H). Mass Spectrum (ESI) m/e=562 (M+1).

### Example 40

N-Cyclopropylmethyl-N-(2-{1-hydroxy-3-[4-(2-methylpropane-1-sulfonyl)-phenyl]-1-trifluoromethyl-prop-2-ynyl}-4-methyl-phenyl)-benzenesulfonamide (40). ¹H NMR (CDCl₃) δ 0.10-0.12 (m, 2H), 0.44-0.46 (m, 2H), 0.88 (d, J=6.3 Hz, 6H), 0.94-0.96 (m, 1H), 1.56-1.58 (m, 2H), 1.57-1.63 (m, 1H), 3.07-3.12 (m, 2H), 3.57 (dd, J=13.9 Hz, J=7.0 Hz, 1H), 3.65 (dd, J=14.0 Hz, J=7.3 Hz, 1H), 7.13 (s, 1H), 7.23-7.25 (m, 3H), 7.32-7.34 (m, 2H), 7.46-7.49 (m, 1H), 7.59-7.63 (m, 1H), 7.72-7.74 (m, 2H), 7.87-7.90 (m, 3H), 7.99-8.01 (m, 1H). Mass Spectrum (ESI) m/e=620 (M+1).

### Example 41

N-Cyclopropylmethyl-N-{2-[1-hydroxy-3-(4-methoxyphenyl)-1-trifluoromethyl-prop-2-ynyl]-phenyl}-benzenesulfonamide (41). ¹H NMR (CDCl₃) δ 0.08-0.12 (m, 2H), 0.42-0.46 (m, 2H), 0.92-0.96 (m, 1H), 3.54 (dd, J=14.0 Hz, J=7.0 Hz, 1H), 3.67 (dd, J=14.0 Hz, J=7.3 Hz), 3.83 (s, 3H), 6.85-6.89 (m, 2H), 7.12 (s, 1H), 7.22-7.25 (m, 3H), 7.30-7.33 (m, 2H), 7.38-7.41 (m, 1H), 7.47-7.49 (m, 2H), 7.55-7.59 (m, 1H), 7.79-7.82 (m, 1H), 8.10-8.12 (m, 1H). Mass Spectrum (ESI) m/e=516 (M+1).

### Example 42

N-Cyclopropylmethyl-N-{2-[1-hydroxy-3-(3-methanesulfonylphenyl)-1-trifluoromethyl-prop-2-ynyl]-phenyl}-benzenesulfonamide (42). ¹H NMR (CDCl₃) δ 0.10-0.13 (m, 2H), 0.44-0.46 (m, 2H), 0.91-0.95 (m, 1H), 3.08 (s, 3H), 3.57 (dd, J=14.0 Hz, J=7.0 Hz, 1H), 3.65 (dd, 14.0 Hz, J=7.0 Hz, 1H), 7.19 (s, 1H), 7.22-7.24 (m, 3H), 7.32-7.34 (m, 2H), 7.45-7.48 (m, 1H), 7.57-7.62 (m, 2H), 7.81-7.88 (m, 2H), 7.95-7.97 (m, 1H), 8.02-8.04 (m, 1H), 8.11-8.12 (m, 1H). Mass Spectrum (ESI) m/e=564 (M+1).

### Example 43

N-Cyclopropylmethyl-N-{2-[1-hydroxy-3-(4-methanesulfonylphenyl)-1-trifluoromethyl-prop-2-ynyl]-phenyl}-benzenesulfonamide (43). ¹H NMR (CDCl₃) δ. Mass Spectrum (ESI) m/e=564 (M+1).

### Example 44

4-{3-[2-(Benzenesulfonyl-cyclopropylmethylamino)-phenyl]-4,4,4-trifluoro-3-hydroxy-but-1-ynyl}-N-methyl-N-propyl-benzenesulfonamide (44). ¹H NMR (CDCl₃) δ 0.10-0.13 (m, 2H), 0.44-0.46 (m, 2H), 0.90-0.93 (m, 1H), 0.93 (t, J=7.4 Hz, 3H), 1.53-1.58 (m, 2H), 2.73 (s, 3H), 2.97 (t, J=7.2 Hz, 2H), 3.56 (dd, J=14.0 Hz, J=7.0 Hz, 1H), 3.65 (dd, J=14.0 Hz, J=7.3 Hz, 1H), 7.13 (s, 1H), 7.22-7.24 (m, 3H), 7.32-7.33 (m, 2H), 7.46-7.48 (m, 1H), 7.57-7.60 (m, 1H), 7.66-7.68 (m, 2H), 7.75-7.77 (m, 2H), 7.86-7.88 (m, 1H), 8.00-8.02 (m, 1H). Mass Spectrum (ESI) m/e=621 (M+1).

### Example 45

4-{3-[2-(Benzenesulfonyl-cyclopropylmethylamino)-phenyl]-4,4,4-trifluoro-3-hydroxy-but-1-ynyl}-N-methyl-benzamide (45). ¹H NMR (CDCl₃) δ 0.09-0.12 (m, 2H), 0.43-0.45 (m, 2H), 0.92-0.94 (m, 1H), 3.03 (d, J=4.9 Hz, 3H), 3.55 (dd, J=14.0 Hz, J=7.0 Hz, 1H), 3.64 (dd, J=14.0 Hz, J=7.2 Hz, 1H), 6.13(s, 1H), 7.15 (s, 1H), 7.21-7.24 (m, 3H), 7.31-7.33 (m, 2H), 7.41-7.44 (m, 1H), 7.57-7.60(m, 1H), 7.58-7.60 (m, 2H), 7.73-7.75 (m, 2H), 7.83-7.85 (m, 1H), 8.04-8.06 (m, 1H). Mass Spectrum (ESI) m/e=543.

### Example 47

4-{3-[2-(Benzenesulfonyl-cyclopropylmethylamino)-phenyl]-4,4,4-trifluoro-3-hydroxy-but-1-ynyl}-N-(2-dimethylamino-ethyl)-N-methyl-benzamide (47). ¹H NMR (CDCl₃) δ 0.07-0.12 (m, 2H), 0.43-0.45 (m, 2H), 0.90-1.0 (m, 1H), 2.93 (br s, 6H), 3.13 (br s, 3H), 3.30-3.40 (m, 2H), 3.55 (dd, J=14.0 Hz, J=7.0 Hz, 1H), 3.64 (dd, J=14.0 Hz, J=7.4 Hz, 1H), 4.0-4.1 (m, 2H), 7.14 (s, 1H), 7.21-7.23 (m, 2H), 7.31-7.33 (m, 3H), 7.43 (t, J=7.4 Hz, 1H), 7.5-7.6 (m, 4H), 7.84 (d, J=8.0 Hz, 1H), 8.04 (d, J=8.0 Hz, 1H). Mass Spectrum (ESI) m/e=614 (M+1).

### Example 48

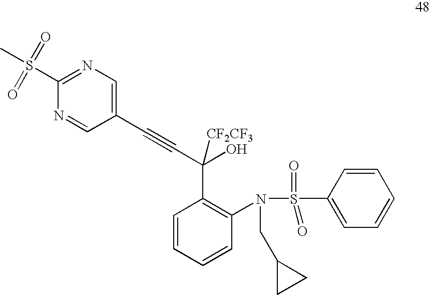

### N-Cyclopropylmethyl-N-{2-[1-hydroxy-3-(4-methanesulfonylphenyl)-1-pentafluoroethyl-prop-2-ynyl]-phenyl}-benzenesulfonamide (48)

Step A. N-Cyclopropylmethyl-N-(2-trifluoroacetyl-phenyl)-benzenesulfonamide. To a solution of 1.0 g (2.73 mmol) of N-(2-bromophenyl)-N-cyclopropylmethyl-benzene sulfonamide in 10 mL of THF at −78° C. was added 3.3 mL (5.6 mmol) of t-butyllithium (1.7 M solution in pentane) dropwise. The mixture was stirred for 20 min at −78° C. and 0.63 g (3.28 mmol) of ethyl pentafluoropropionate was added in a single portion. The resultant mixture was stirred at −78° C. for 15 min, warmed to 0° C. and stirred for an additional 5 min. The reaction mixture was quenched with saturated aqueous ammonium chloride and extracted with ether. The organic layer was washed with brine, dried over MgSO₄, filtered, and the filtrate was concentrated. The residue was purified by chromatography on silica gel (hexanes:EtOAc, 7:3) to give the title compound. ¹H NMR (CDCl₃) δ 0.02 (m, 2H), 0.42 (m, 2H), 1.01 (m, 1H), 3.52 (m, 2H), 7.00-7.82 (m, 9H). Mass Spectrum (ESI) m/e=434 (M⁺+H₃O).

Step B. N-Cyclopropylmethyl-N-{2-[1-hydroxy-3-(4-methanesulfonylphenyl)-1-pentafluoroethyl-prop-2-ynyl]-phenyl}-benzenesulfonamide. The title compound was prepared as described in Example 1, Step D. ¹H NMR (CDCl₃) δ 0.15 (m, 2H), 0.49 (m, 2H), 0.95 (m, 1H), 3.05 (s, 3H), 3.46 (dd, J=7.7 Hz, 14.0 Hz, 1H), 3.61 (dd, J=7.7 Hz, 14.0 Hz, 1H), 6.76 (d, J=8.0 Hz, 1H), 6.94 (d, J=2.9 Hz, 1H), 7.24-7.93 (m, 12H). Mass Spectrum (ESI) m/e=614 (M+1).

### Example 49

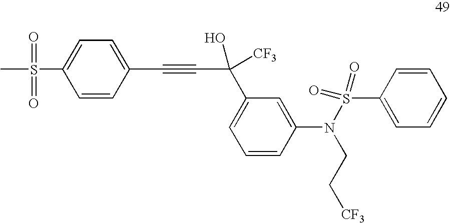

### N-{3-[1-Hydroxy-3-(4-methanesulfonylphenyl)-1-trifluoromethyl-prop-2-ynyl]-phenyl}-N-(3,3,3-trifluoropropyl)-benzenesulfonamide (49)

Step A. N-(3-Bromophenyl)-benzenesulfonamide. To a solution of 9.7 mL (76.0 mmol) of benzenesulfonyl chloride in 75 mL of dichloromethane at 0° C. was added 11.4 mL (141.0 mmol) of pyridine and 10.0 mL (98%, 90.0 mmol) of 3-bromoaniline sequentially. The mixture was allowed to gradually warm up to room temperature overnight (17 h) and diluted with dichloromethane. The resultant mixture was washed with saturated aqueous ammonium chloride, 1 M citric acid solution (2×), saturated aqueous sodium bicarbonate and brine, dried over Na₂SO₄, filtered, and the filtrate was concentrated to give the title compound. ¹H-NMR (CDCl₃) δ 3.78 (br s, 1H), 6.98-7.05 (m, 1H), 7.09 (t, J=8.0 Hz, 1H), 7.19-7.28 (m, 2H), 7.46 (t, J=5.1 Hz, 2H), 7.56 (t, J=7.4 Hz, 1H), 7.81 (d, J=7.4 Hz, 2H). Mass Spectrum (ESI) m/e=312.0 (M+1), 329.0 (M+18).

Step B. N-(3-Bromophenyl)-N-(3,3,3-trifluoropropyl)-benzenesulfonamide. To a suspension of 757 mg (18.9 mmol) of NaH (60% dispersion in oil) in 13.5 mL of DMF was added a solution of 4.91 g (15.7 mmol) of N-(3-bromophenyl)-benzenesulfonamide in 8.5 mL of DMF. The mixture was stirred for 30 min. 1,1,1-Trifluopropyl-3-iodopropane (1.95 mL, 16.6 mmol) was added and the resultant mixture was heated to 50° C. and stirred for 20 h at this temperature. The reaction mixture was cooled to room temperature, quenched with saturated aqueous ammonium chloride and extracted with ethyl acetate. The organic layer was washed with saturated aqueous sodium bicarbonate and brine, dried over Na₂SO₄, filtered, and the filtrate was concentrated. The residue was purified by chromatography on silica gel (hexanes:EtOAc, 19:1) to give the title compound.

Step C. N-(3-Trifluoroacetyl-phenyl)-N-(3,3,3-trifluoropropyl)-benzenesulfonamide. To a solution of 405 mg (0.99 mmol) of N-(3-bromophenyl)-N-(3,3,3-trifluoropropyl)-benzenesulfonamide in 10 mL of THF at −78° C. was added dropwise 416 μL (1.04 mmol) of n-BuLi (2.5 M solution in hexanes). The mixture was stirred at −78° C. for 10 min. Ethyl trifluoroacetate (130 μL, 1.09 mmol) was added and the resultant mixture was stirred at −78° C. for 25 min. The reaction mixture was quenched with saturated aqueous ammonium chloride and extracted with ethyl acetate (3×). The combined organic layers were washed with brine, dried over Na₂SO₄, filtered, and the filtrate was concentrated. The residue was purified by chromatography on silica gel (hexanes:EtOAc, 4:1) to give the title compound.

Step D. N-{3-[1-Hydroxy-3-(4-methanesulfonylphenyl)-1-trifluoromethyl-prop-2-ynyl]-phenyl}-N-(3,3,3-trifluoropropyl)-benzenesulfonamide. To a solution of 39 mg (0.22 mmol) of 1-ethynyl-4-methanesulfonylbenzene (Example 1, Step A) in 4 mL of THF at −78° C. was added dropwise 82 μL (0.21 mmol) of n-BuLi (2.5 M solution in hexanes). The mixture was stirred at −78° C. for 1 h. Cerium(III)chloride (540 μL, 0.11 mmol, 0.2 M suspension in THF) was added. After an additional 30 min at −78° C., a solution of 46 mg (0.11 mmol) of N-(3-trifluoroacetyl-phenyl)-N-(3,3,3-trifluoropropyl)-benzenesulfonamide in 3 mL of THF was added and the resultant mixture was stirred at −78° C. for 1 h. The reaction mixture was quenched with saturated aqueous ammonium chloride and extracted with ethyl acetate (3×). The combined organic layers were dried over Na₂SO₄, filtered, and the filtrate was concentrated. The residue was purified by chromatography on silica gel (hexanes:EtOAc, 13:7) to give the title compound. ¹H-NMR (CDCl₃) δ 2.32-2.45 (m, 2H), 3.06 (s, 3H), 3.75-3.84 (m, 2H), 3.87 (s, 1H), 7.19 (d, J=8.0 Hz, 1H), 7.41-7.48 (m, 4H), 7.53-7.59 (m, 3H), 7.62 (d, J=8.1 Hz, 2H), 7.76 (d, J=7.9 Hz, 1H), 7.91 (d, J=8.2 Hz, 2H). Mass Spectrum (ESI) m/e=606.1 (M+1), 623.0 (M+18), 628.0 (M+23).

The compounds listed in the following table were prepared according to the procedure described in Example 49.

TABLE 3

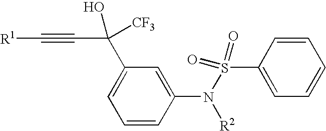

| Compound | R¹ | R² |
|---|---|---|
| 50 | 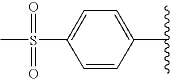 | ⁱPr |
| 51 | 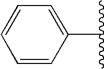 | ⁱBu |
| 52 | 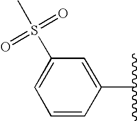 | ⁱBu |
| 54 | 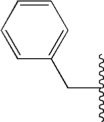 | ⁱBu |
| 55 | 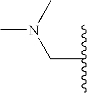 | ⁱBu |
| 56 | 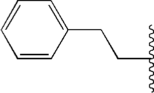 | ⁱBu |
| 57 | 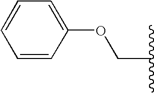 | ⁱBu |
| 58 | 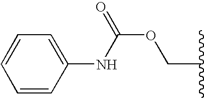 | ⁱBu |
| 59 | 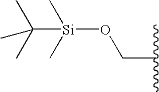 | ⁱBu |
| 60 | 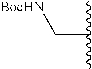 | ⁱBu |
| 61 | 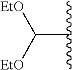 | ⁱBu |
| 62 | 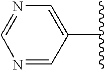 | 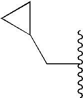 |
| 63 | 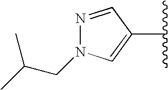 | 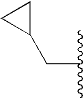 |
| 64 | 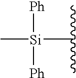 | 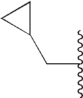 |

### Example 50

N-{3-[1-Hydroxy-3-(4-methanesulfonylphenyl)-1-trifluoromethyl-prop-2-ynyl]-phenyl}-N-isopropyl-benzenesulfonamide (50). ¹H-NMR (CDCl₃) δ 1.04 (t, J=7.1 Hz, 6H), 3.07 (s, 3H), 3.44 (s, 1H), 4.63 (quintet, J=7.0 Hz, 1H), 7.20 (d, J=8.8 Hz, 1H), 7.40-7.47 (m, 4H), 7.51 (dt, J=6.6 Hz, 1.3 Hz, 1H), 7.66 (d, J=8.0 Hz, 2H), 7.71-7.76 (m, 2H), 7.79 (d, J=8.0 Hz, 1H), 7.95 (d, J=8.2 Hz, 2H). Mass Spectrum (ESI) m/e=569.0 (M+18).

### Example 51

N-[3-(1-Hydroxy-3-phenyl-1-trifluoromethyl-prop-2-ynyl)-phenyl]-N-isobutyl-benzenesulfonamide (51). ¹H NMR (CDCl₃) δ 0.91 (m, 6H), 1.58 (m, 1H), 2.95 (bs, 1H), 5.34 (m, 2H), 7.26-7.55 (m, 13H), 7.74 (d, J=8.0 Hz, 1H). Mass Spectrum (ESI) m/e=488 (M+1).

### Example 52

N-{3-[1-Hydroxy-3-(3-methanesulfonylphenyl)-1-trifluoromethyl-prop-2-ynyl]-phenyl}-N-isobutyl-benzenesulfonamide (52). ¹H NMR (CDCl₃) δ 0.91 (m, 6H), 1.59 (m, 1H), 3.09 (s, 3H), 3.23 (bs, 1H), 3.34 (m, 2H), 7.20-7.79 (m, 11H), 7.98 (d, J=8.2 Hz, 1H), 8.09 (s, 1H). Mass Spectrum (ESI) m/e=566 (M+1).

### Example 61

N-[3-(4,4-Diethoxy-1-hydroxy-1-trifluoromethyl-but-2-ynyl]-phenyl}-N-isobutyl-benzenesulfonamide (61). ¹H NMR (CDCl₃) δ 0.83 (6H, d, Me₂), 1.50 (1H, m, CHMe₂), 3.25 (2H, d, CH₂N), 3.71-3.46 (4H, m, 2×OCH₂CH₃), 7.60-7.12 (9H, m, Ar). Mass Spectrum m/e=514 (M+1).

### Example 62

N-Cyclopropylmethyl-N-[3-(1-hydroxy-3-pyrimidin-5-yl-1-trifluoromethyl-prop-2-ynyl)-phenyl]-benzenesulfonamide (62). ¹H NMR (CDCl₃) δ 0.09-0.10 (m, 2H), 0.39-0.41 (m, 2H), 0.84-0.86 (m, 1H), 3.44-3.47 (m, 2H), 4.83 (s, 1H), 7.25-7.26 (m, 1H), 7.41-7.45 (m, 3H), 7.49-7.52 (m, 2H), 7.59-7.61 (m, 2H), 7.73 (d, J=7.8 Hz, 1H), 8.86 (s, 2H), 9.2 (s, 1H). Mass Spectrum (ESI) m/e=488 (M+1).

### Example 63

N-Cyclopropylmethyl-N-{3-[1-hydroxy-3-(1-isobutyl-1H pyrazol-3-yl)-1-trifluoromethyl-prop-2-ynyl]-phenyl}-benzenesulfonamide (63). ¹H NMR (CDCl₃) δ 0.08-0.09 (m, 2H), 0.39-0.41 (m, 2H), 0.84-0.92 (m, 7H), 2.20 (m, 1H), 3.44 (d, J=7.1 Hz, 2H), 3.65 (s, 1H), 3.90 (d, J=7.3 Hz), 7.29 (s, 1H), 7.38-7.44 (m, 4H), 7.50-7.61 (m, 5H), 7.73 (d, J=7.94 Hz, 1H). Mass Spectrum (ESI) m/e=532.

### Example 64

N-Cyclopropylmethyl-N-{3-[1-hydroxy-3-(methyldiphenylsilanyl)-1-trifluoromethyl-prop-2-ynyl]-phenyl}-benzenesulfonamide (64). ¹H NMR (CDCl₃) δ 0.10 (m, 2H), 0.41 (m, 2H), 0.79(s, 3H), 0.87 (m 1H), 3.17 (s, 1H), 3.38 (dd, J=7.0 Hz, 13.5 Hz, 1 h), 3.50 (dd, J=7.0 Hz, 13.5 Hz, 1H), 7.33-7.77 9 m 19H). Mass Spectrum (ESI) m/e=624 (M+18).

The compounds listed in the following table were prepared according to the procedure described in Example 49.

TABLE 4

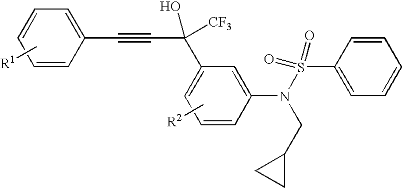

| Compound | R¹ | R² |
|---|---|---|
| 65 | 4-MeS | H |
| 67 | 4-MeSO₂ | 4-Me |
| 68 | 3-MeSO₂ | H |
| 65 | 4-MeS | H |
| 67 | 4-MeSO₂ | 4-Me |
| 68 | 3-MeSO₂ | H |
| 69 | 4-(CH₃)₂CHCH₂CH₂SO₂ | H |
| 70 | 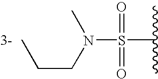 | 4-Me |
| 72 | 4-NHAc | 4-Me |

### Example 65

N-Cyclopropylmethyl-N-{3-[1-hydroxy-3-(4-methylsulfanylphenyl)-1-trifluoromethyl-prop-2-ynyl]-phenyl}-benzenesulfonamide (65). ¹H NMR (CDCl₃) δ 0.03-0.15 (m, 2H), 0.35-0.44 (m, 2H), 0.78-0.91 (m, 1H), 2.50 (s, 3H), 3.20 (s, 1H), 3.38-3.49 (m, 2H), 7.20 (d, J=8.5 Hz, 2H), 7.30 (d, J=7.9 Hz, 1H), 7.35-7.52 (m, 7H), 7.57-7.62 (m, 2H), 7.74 (d, J=7.9 Hz, 1H). Mass Spectrum (ESI) m/e=532.2 (M+1), 554.0 (M+23).

### Example 67

N-Cyclopropylmethyl-N-{3-[1-hydroxy-3-(4-methylsulfonylphenyl)-1-trifluoromethyl-prop-2-ynyl]-4-methylphenyl}-benzenesulfonamide (67). ¹H NMR (CDCl₃) δ 0.09 (m, 2H), 0.39 (m, 2H), 0.85 (m, 1H), 2.66 (s, 3H), 3.07 (s, 3H), 3.17 (s, 1H), 3.41 (d, J=7.0 Hz, 2H), 7.09-7.63 (m, 8H), 7.67 (d, J=8.1 Hz, 2H), 7.94 (d, J=8.1 Hz, 2H). Mass Spectrum (ESI) m/e=578 (M+1).

### Example 68

N-Cyclopropylmethyl-N-{3-[1-hydroxy-3-(3-methylsulfonylphenyl)-1-trifluoromethyl-prop-2-ynyl]-phenyl}-benzenesulfonamide (68). ¹H NMR (CDCl₃) δ 0.09 (m, 2H), 0.40 (m, 2H), 0.85 (m, 1H), 3.09 (s, 3H), 3.44 (m, 2H), 3.48 (s, 1H), 7.22-8.08 (m, 13H). Mass Spectrum (ESI) m/e=564 (M+1).

### Example 69

N-Cyclopropylmethyl-N-(3-{1-hydroxy-3-[4-(3-methylbutane-1-sulfonyl)phenyl]-1-trifluoromethyl-prop-2-ynyl}-phenyl)-benzenesulfonamide (69). ¹H NMR (CDCl₃) δ 0.09 (m, 2H), 0.39 (m, 2H), 0.85 (m, 1H), 0.88 (d, J=6.3 Hz, 6H), 1.59 (m, 3H), 3.09 (m, 2H), 3.27 (bs, 1H), 3.44 (d, J=7.0 Hz, 2H), 7.23-7.74 (m, 11H), 7.90 (d, J=8.5 Hz, 2H). Mass Spectrum (ESI) m/e=620 (M+1).

### Example 70

3-{3-[5-(Benzenesulfonyl-cyclopropylmethylamino)-2-methyl-phenyl]-4,4,4-trifluoro-3-hydroxy-but-1-ynyl}-N-methyl-N-propyl-benzenesulfonamide (70). ¹H NMR (CDCl₃) δ 0.25 (m, 2H), 0.55 (m, 2H), 1.03 (m, 1H), 1.09 (t, J=7.4 Hz, 3H), 1.47 (s, 1H), 1.73 (m, 2H), 2.82 (s, 3H), 2.91 9 s, 3H), 3.15 (t, J=7.3 Hz, 2H), 3.57 (d, J=7.2 Hz, 2H), 7.25 (d, J=2.2 Hz, 1H), 7.28 (d, J=2.2 Hz, 1H), 7.35-7.95 (m, 10H). Mass Spectrum (ESI) m/e=635 (M+1).

### Example 72

4-{3-[5-(Benzenesulfonyl-cyclopropylmethylamino)-2-methyl-phenyl]-4,4,4-trifluoro-3-hydroxy-but-1-ynyl}-phenyl)-acetamide (72). ¹H NMR (CDCl₃) δ 0.25 (m, 2H), 0.55 (m, 2H), 1.00 (m, 1H), 2.40 (s, 3H), 2.83 (s, 3H), 3.57 (d, J=7.0 Hz, 2H), 4.62 (br s, 2H), 7.29-7.68 (m 10H), 7.78 (d, J=8.0 Hz, 2H). Mass Spectrum (ESI) m/e=557 (M+1).

### Example 73

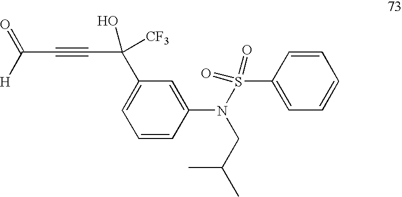

N-[3-(1-Hydroxy-4-oxo-1-trifluoromethyl-but-2-ynyl)-phenyl]-N-isobutyl-benzenesulfonamide (73). A solution of 785 mg (1.5 mmol) of N-[3-(4,4-diethoxy-1-hydroxy-1-trifluoromethyl-but-2-ynyl]-phenyl}-N-isobutyl-benzenesulfonamide (Example 61) and 1.44 g of p-toluenesulfonic acid monohydrate (7.5 mmol) in 30 mL of acetone was heated to reflux for 2 h and cooled to room temperature. The reaction was concentrated under reduced pressure and the residue was purified by chromatography on silica gel (hexanes:EtOAc, 7:3) to give the title compound. ¹H NMR (CDCl₃) δ 0.85 (6H, m, CHMe₂), 1.50 (1H, m, CHMe₂), 3.28 (2H, m, CH₂N), 7.61-7.13 (9H, m, Ar), 9.25 (1H, s, CHO).

### Example 74

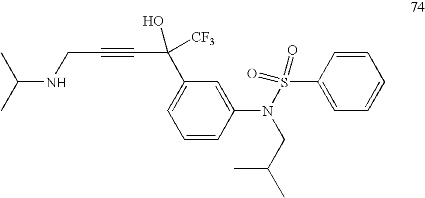

N-[3-(1-Hydroxy-4-isopropylamino-1-trifluoromethyl-but-2-ynyl)-phenyl]-N-isobutyl-benzenesulfonamide (74). 650 mg (1.48 mmol) of N-[3-(1-hydroxy-4-oxo-1-trifluoromethyl-but-2-ynyl)-phenyl]-N-isobutyl-benzenesulfonamide (Example 73) and 0.2 mL of isopropyl amine (2.35 mmol) were combined in 5 mL of dichloromethane and stirred at room temperature. After 14 h the solution was concentrated under reduced pressure and the residue was stirred in methanol (10 mL) at room temperature. Sodium borohydride (35 mg, 0.92 mmol) was added and the reaction was stirred until evolution of hydrogen had stopped. Water (1 mL) was added and the reaction was concentrated under reduced pressure. The residue was purified by chromatography on silica gel (chloroform: MeOH, 9:1) to give the title compound. ¹H NMR (CDCl₃) δ 0.84 (6H, d, CH₂CHMe₂), 1.02 (6H, d, NHCHMe₂), 1.50 (1H, m, CHMe₂), 2.94 (1H, m, NHCHMe₂), 3.25 (2H, d, CH₂N), 3.46 (2H, s, CCH₂NH), 7.63-7.04 (9H, m, Ar). Mass Spectrum (ESI) m/e=483 (M+1).

### Example 75

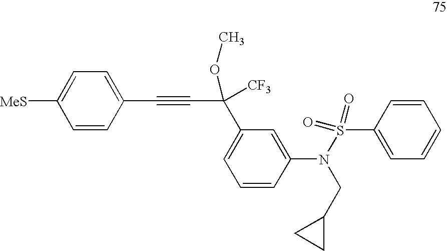

N-Cyclopropylmethyl-N-{3-[1-methoxy-3-(3-methylsulfanylphenyl)-1-trifluoromethyl-prop-2-ynyl]-phenyl}-benzenesulfonamide (75). To a suspension of 21 mg (0.53 mmol) of sodium hydride (60% dispersion in oil) in 2 mL of THF at 0° C. was added a solution of 25 mg (0.05 mmol) of N-cyclopropyl-methyl-N-{3-[1-hydroxy-3-(4-methylsulfanylphenyl)-1-trifluoromethyl-prop-2-ynyl]-phenyl}-benzenesulfonamide (Example 65) in 2 mL of THF. The mixture was warmed to room temperature and stirred for 45 min. Methyl iodide (65 μL, 1.05 mmol) was added and the resultant mixture was stirred for 12 h. The reaction mixture was quenched with water and extracted with ethyl acetate (3×). The combined organic layers were dried over Na₂SO₄, filtered, and the filtrate was concentrated. The residue was purified by chromatography on silica gel (hexanes:EtOAc, 9:1) to give the title compound. ¹H-NMR (CDCl₃) δ 0.06-0.14 (m, 2H), 0.35-0.45 (m, 2H), 0.79-0.92 (m, 1H), 2.51 (s, 3H), 3.26 (s, 3H), 3.43-3.51 (m, 2H), 7.22 (d, J=8.2 Hz, 2H), 7.31-7.54 (m, 8H), 7.60 (d, J=7.4 Hz, 2H), 7.70 (d, J=7.0 Hz, 1H). Mass Spectrum (ESI) m/e=546.0 (M+1), 563.1 (M+18).

### Example 80

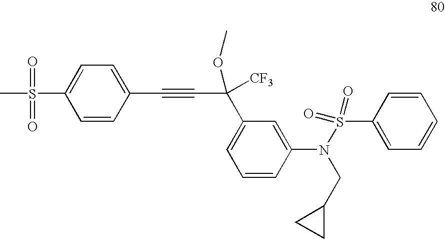

N-Cyclopropylmethyl-N-{3-[1-methoxy-3-(3-methylsulfonylphenyl)-1-trifluoromethyl-prop-2-ynyl]-phenyl}-benzenesulfonamide (80). A slurry of 16 mg (0.03 mmol) of N-Cyclopropylmethyl-N-{3-[1-methoxy-3-(3-methylsulfanylphenyl) 1-trifluoromethyl-prop-2-ynyl]-phenyl}-benzenesulfonamide (Example 75) and 277 mg (0.45 mmol) of Oxone® in 3 mL of MeOH and 1.5 mL of water was stirred at room temperature for 21 h. The reaction mixture was diluted with water and extracted with ethyl acetate (3×). The combined organic layers were dried over Na₂SO₄, filtered, and the filtrate was concentrated to give the title compound. ¹H-NMR (CDCl₃) δ 0.05-0.12 (m, 2H), 0.36-0.44 (m, 2H), 0.78-0.94 (m, 1H), 3.09 (s, 3H), 3.40 (s, 3H), 3.35-3.44 (m, 2H), 7.29 (d, J=7.9 Hz, 1H), 7.35-7.47 (m, 4H), 7.47-7.55 (m, 1H), 7.61 (d, J=7.5 Hz, 2H), 7.68 (d, J=7.6 Hz, 1H), 7.74 (d, J=8.1 Hz, 2H), 7.97 (d, J=8.1 Hz, 2H). Mass Spectrum (ESI) m/e=578.0 (M+1), 595.2 (M+18), 600.1 (M+23).

### Example 85

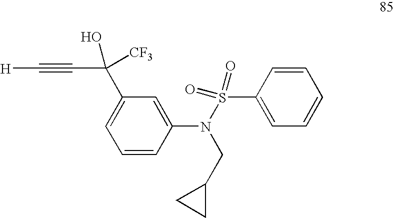

N-Cyclopropylmethyl-N-[3-(1-hydroxy-1-trifluoromethyl-prop-2-ynyl)-phenyl]-benzenesulfonamide (85). To a solution of 0.31 g (0.5 mmol) of N-cyclopropylmethyl-N-{3-[1-hydroxy-3-(methyldiphenylsilanyl)-1-trifluoromethyl-prop-2-ynyl]-phenyl}-benzenesulfonamide (Example 64) in 3 mL of THF was added 0.15 g acetic acid (2.5 mmol) and 0.5 mL of tetrabutyl ammonium fluoride (0.5 mmol; 1 M solution in THF) at room temperature. The resulting mixture was stirred at room temperature for 2.5 h. The reaction mixture was quenched with water and extracted with ethyl acetate (3×). The combined organic layers were washed with saturated aqueous ammonium chloride and brine, dried over Na₂SO₄, filtered, and the filtrate was concentrated. The residue was purified by chromatography on silica gel (hexanes:EtOAc, 7:3) to give the title compound. ¹H NMR (CDCl₃) δ 0.08 (m, 2H), 0.40 (m, 2H), 0.84 (m, 1H), 2.76 (s, 1H), 3.26 (br s, 1H), 3.44 (m, 2H), 7.28-7.63 (m. 8H), 7.69 (d, J=8.1 Hz, 1H). Mass Spectrum m/e=410.0 (M+1).

### Example 86

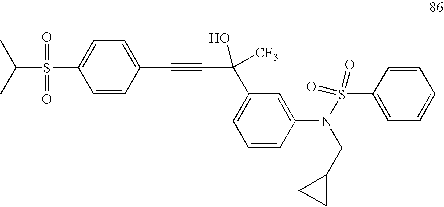

N-Cyclopropylmethyl-N-(3-{1-hydroxy-3-[4-(propane-2-sulfonyl)phenyl]-1-trifluoromethyl-prop-2-ynyl}-phenyl)-benzenesulfonamide (86). A mixture of 210 mg of 1-iodo-4-isopropylsulfonyl-benzene (0.68 mmol), 7.3 mg of palladium on carbon (10% Pd, 0.01 mmol), 2.6 mg of copper (I) iodide (0.01 mmol), 5.4 mg of triphenylphosphine (0.02 mmol) and 120 mg of K₂CO₃ (0.86 mmol) in 2 mL of DME and 2 mL of water was deairated by purging with nitrogen for 30 min. A solution of 140 mg (0.34 mmol) of N-cyclopropylmethyl-N-[3-(1-hydroxy-1-trifluoromethyl-prop-2-ynyl)-phenyl]benzenesulfonamide (Example 85) in 1 mL of DME was added and the resulting mixture was stirred at 65° C. for 16 h. The reaction mixture was cooled to room temperature and poured into 60 mL of ethyl acetate. The catalyst was removed by filtration through a pad of celite and the filtrate was washed with saturated aqueous ammonium chloride and brine, dried over Na₂SO₄, filtered, and the filtrate was concentrated. The residue was purified by chromatography on silica gel (hexanes:EtOAc, 7:3) to give the title compound. ¹H NMR (CDCl₃) δ 0.25 (m, 2H), 0.57 (m, 2H), 1.02 (m, 1H), 1.46 (d, J=7.0 Hz, 6H), 3.37 (m, 1H), 3.39 (br s, 1H), 3.61 (d, J=7.2 Hz, 2H), 7.4-7.84 (m, 10H), 7.89 (d, J=8.0 Hz, 1H), 8.04 (d, J=8.6 Hz, 2H). Mass Spectrum (ESI) m/e=592 (M+1).

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A compound having the formula: 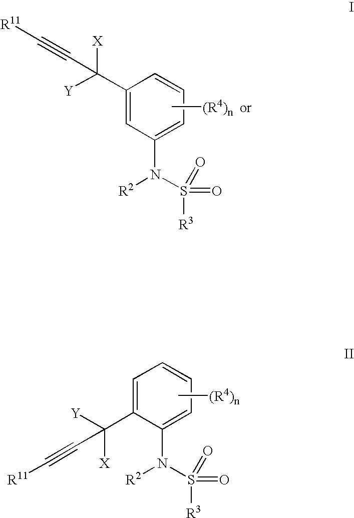
or a pharmaceutically acceptable salt or prodrug thereof, wherein
R¹¹ is a member selected from the group consisting of hydrogen, halogen, nitro, cyano, R¹², OR¹², SR¹², NHR¹², N(R¹²)₂, (C₅-C₈)cycloalkenyl, COR¹², CO₂R¹², CONHR¹², CON(R¹²)₂, C═N—NR¹², aryl(C₁-C₄)alkyl, heteroaryl, heteroaryl(C₁-C₄)alkyl, (C₄-C₈)cycloalkyl(C₁-C₄)alkyl and hetero(C₄-C₈)cycloalkyl(C₁-C₄)alkyl; wherein each R¹² is (C₁-C₈)alkyl, (C₃-C₈)alkenyl, (C₃-C₈)alkynyl, (C₂-C₈)heteroalkyl, halo(C₁-C₈)alkyl, (C₄-C₈)cycloalkyl, aryl or two R¹² groups attached to the same nitrogen atom are combined to form a five- to eight-membered ring and any alkyl portions of R¹¹ are optionally substituted with from one to three substituents independently selected from the group consisting of halogen, OR¹³, NHSO₂R¹⁴ and NHC(O)R¹³, and any aryl or heteroaryl portions of R¹¹ are optionally substituted with from one to five substituents independently selected from the group consisting of halogen, cyano, nitro, R¹⁴, OR¹³, SR¹³, N(R¹³)₂, CO₂R¹³, CON(R¹³)₂, C(O)R¹³, SO₂R¹³, SO₂N(R¹³)₂, NHSO₂R¹⁴, NHC(O)R¹³, phenyl, phenyl(C₁-C₈)alkyl and phenyl(C₂-C₈)heteroalkyl; wherein each R¹³ is independently selected from H, (C₁-C₈)alkyl, (C₃-C₈)alkenyl, (C₃-C₈)alkynyl, (C₂-C₈)heteroalkyl and halo(C₁-C₈)alkyl and each R¹⁴ is independently selected from (C₁-C₈)alkyl, (C₃-C₈)alkenyl, (C₃-C₈)alkynyl, (C₂-C₈)heteroalkyl and halo(C₁-C₈)alkyl;
X is a member selected from the group consisting of H, NH₂, NHR¹⁵, NHSO₂R¹⁵, OH and OR′, wherein R¹⁵ is (C₁-C₈)alkyl, (C₃-C₈)alkenyl, (C₃-C₈)alkynyl, (C₂-C₈)heteroalkyl or halo(C₁-C₈)alkyl and R′ is (C₁-C₈)alkyl, (C₃-C₈)alkenyl, (C₃-C₈)alkynyl, (C₂-C₈)heteroalkyl, halo(C₁-C₈)alkyl, aryl(C₁-C₄)alkyl, heterocyclo(C₅-C₈)alkyl, (C₁-C₄)alkylsulfonyl, arylsulfonyl, (C₁-C₄)alkylcarbonyl or (C₁-C₄)alkylsilyl;
Y is fluoro(C₁-C₄)alkyl;
R² is a member selected from the group consisting of H, (C₁-C₈)alkyl, (C₂-C₈)heteroalkyl, (C₃-C₈)alkenyl, (C₃-C₈)alkynyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino, or optionally, R² and R⁴ are combined to form a five- to seven-membered fused ring containing the nitrogen atom to which R² is attached and from 0 to 2 additional heteroatoms selected from N, O and S;
R³ is a member selected from the group consisting of aryl and heteroaryl, said aryl or heteroaryl group being optionally substituted with from one to five substituents independently selected from the group consisting of halogen, cyano, nitro, R¹⁶, OR¹⁶, SR¹⁶, COR¹⁶, CO₂R¹⁶, NHR¹⁶, N(R¹⁶)₂, CONHR¹⁶, CON(R¹⁶)₂, NHSO₂R¹⁶, NHC(O)R¹⁶, phenyl, phenyl(C₁-C₈)alkyl, and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring;
the subscript n is an integer of from 0 to 3; and
each R⁴ is independently selected from the group consisting of halogen, cyano, nitro, R¹⁷, OR¹⁷, SR¹⁷, COR¹⁷, CO₂R¹⁷, N(R¹⁷)₂ and CON(R¹⁷)₂, wherein each R¹⁷ is independently selected from H, (C₁-C₈)alkyl, (C₃-C₈)alkenyl, (C₃-C₈)alkynyl, (C₂-C₈)heteroalkyl and halo(C₁-C₈)alkyl, or two R¹⁷ groups attached to the same nitrogen atom are combined to form a five- to eight-membered ring.

2. A compound of claim 1, wherein X is OH.

3. A compound of claim 2, wherein R¹¹ is phenyl, optionally substituted with from one to two substituents independently selected from the group consisting of halogen, (C₁-C₈)alkyl, (C₂-C₈)heteroalkyl, halo(C₁-C₈)alkyl, phenyl(C₁-C₆)alkyl and phenyl(C₂-C₆)heteroalkyl.

4. A compound of claim 3, wherein R² is selected from the group consisting of H, (C₁-C₈)alkyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino.

5. A compound of claim 4, wherein R³ is a member selected from the group consisting of phenyl, pyridyl, thienyl and thiazolyl, optionally substituted with from one to five substituents independently selected from the group consisting of halogen, cyano, nitro, R¹⁶, OR¹⁶, SR¹⁶, COR¹⁶, CO₂R¹⁶, NHR¹⁶, N(R¹⁶)₂, CONHR¹⁶, CON(R¹⁶)₂, NHSO₂R¹⁶, NHC(O)R¹⁶, phenyl, phenyl(C₁-C₈)alkyl, and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring.

6. A compound of claim 5, wherein the subscript n is an integer of from 0 to 2, and each R⁴ is independently selected from the group consisting of halogen, (C₁-C₈)alkyl and halo(C₁-C₈)alkyl.

7. A compound of claim 6, wherein R² is selected from the group consisting of H, (C₁-C₈)alkyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino.

8. A compound of claim 7, wherein R³ is a member selected from the group consisting of phenyl, pyridyl, thienyl and thiazolyl, optionally substituted with from one to five substituents independently selected from the group consisting of halogen, cyano, nitro, R¹⁶, OR¹⁶, SR¹⁶, COR¹⁶, CO₂R¹⁶, NHR¹⁶, N(R¹⁶)₂, CONHR⁶, CON(R¹⁶)₂, NHSO₂R¹⁶, NHC(O)R¹⁶, phenyl, phenyl(C₁-C₈)alkyl, and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring.

9. A compound of claim 8, wherein the subscript n is an integer of from 0 to 2, and each R⁴ is independently selected from the group consisting of halogen, (C₁-C₈)alkyl and halo(C₁-C₈)alkyl.

10. A pharmaceutical composition comprising a pharmaceutically acceptable excipient and a compound having the formula: 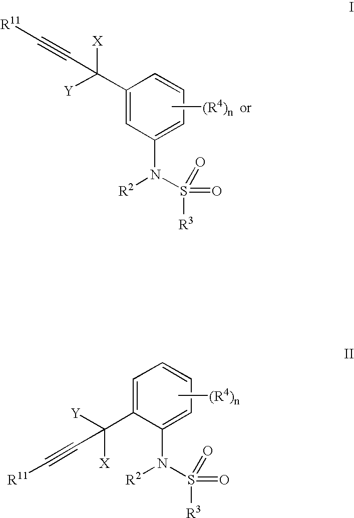
or a pharmaceutically acceptable salt or prodrug thereof, wherein
R¹¹ is a member selected from the group consisting of hydrogen, halogen, nitro, cyano, R¹², OR¹², SR¹², NHR¹², N(R¹²)₂, (C₅-C₈)cycloalkenyl, COR¹², CO₂R¹², CONHR¹², CON(R¹²)₂, C═N—NR¹², aryl(C₁-C₄)alkyl, heteroaryl, heteroaryl(C₁-C₄)alkyl, (C₄-C₈)cycloalkyl(C₁-C₄)alkyl and hetero(C₄-C₈)cycloalkyl(C₁-C₄)alkyl; wherein each R¹² is (C₁-C₈)alkyl, (C₃-C₈)alkenyl, (C₃-C₈)alkynyl, (C₂-C₈)heteroalkyl, halo(C₁-C₈)alkyl, (C₄-C₈)cycloalkyl, aryl or two R¹² groups attached to the same nitrogen atom are combined to form a five- to eight-membered ring and any alkyl portions of R¹¹ are optionally substituted with from one to three substituents independently selected from the group consisting of halogen, OR¹³, NHSO₂R¹⁴ and NHC(O)R¹³, and any aryl or heteroaryl portions of R¹¹ are optionally substituted with from one to five substituents independently selected from the group consisting of halogen, cyano, nitro, R¹⁴, OR¹³, SR¹³, N(R¹³)₂, CO₂R¹³, CON(R¹³)₂, C(O)R¹³, SO₂R¹³, SO₂N(R¹³)₂, NHSO₂R¹⁴, NHC(O)R¹³, phenyl, phenyl(C₁-C₈)alkyl and phenyl(C₂-C₈)heteroalkyl; wherein each R¹³ is independently selected from H, (C₁-C₈)alkyl, (C₃-C₈)alkenyl, (C₃-C₈)alkynyl, (C₂-C₈)heteroalkyl and halo(C₁-C₈)alkyl and each R¹⁴ is independently selected from (C₁-C₈)alkyl, (C₃-C₈)alkenyl, (C₃-C₈)alkynyl, (C₂-C₈)heteroalkyl and halo(C₁-C₈)alkyl;
X is a member selected from the group consisting of H, NH₂, NHR¹⁵, NHSO₂R¹⁵, OH and OR′, wherein R¹⁵ is (C₁-C₈)alkyl, (C₃-C₈)alkenyl, (C₃-C₈)alkynyl, (C₂-C₈)heteroalkyl or halo(C₁-C₈)alkyl and R′ is (C₁-C₈)alkyl, (C₃-C₈)alkenyl, (C₃-C₈)alkynyl, (C₂-C₈)heteroalkyl, halo(C₁-C₈)alkyl, aryl(C₁-C₄)alkyl, heterocyclo(C₅-C₈)alkyl, (C₁-C₄)alkylsulfonyl, arylsulfonyl, (C₁-C₄)alkylcarbonyl or (C₁-C₄)alkylsilyl;
Y is fluoro(C₁-C₄)alkyl;
R² is a member selected from the group consisting of H, (C₁-C₈)alkyl, (C₂-C₈)heteroalkyl, (C₃-C₈)alkenyl, (C₃-C₈)alkynyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino, or optionally, R² and R⁴ are combined to form a five- to seven-membered fused ring containing the nitrogen atom to which R² is attached and from 0 to 2 additional heteroatoms selected from N, O and S;
R³ is a member selected from the group consisting of aryl and heteroaryl, said aryl or heteroaryl group being optionally substituted with from one to five substituents independently selected from the group consisting of halogen, cyano, nitro, R¹⁶, OR¹⁶, SR¹⁶, COR¹⁶, CO₂R¹⁶, NHR¹⁶, N(R¹⁶)₂, CONHR¹⁶, CON(R¹⁶)₂, NHSO₂R¹⁶, NHC(O)R¹⁶, phenyl, phenyl(C₁-C₈)alkyl, and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring;
the subscript n is an integer of from 0 to 3; and
each R⁴ is independently selected from the group consisting of halogen, cyano, nitro, R¹⁷, OR¹⁷, SR¹⁷, COR¹⁷, CO₂R¹⁷, N(R¹⁷)₂ and CON(R¹⁷)₂, wherein each R¹⁷ is independently selected from H, (C₁-C₈)alkyl, (C₃-C₈)alkenyl, (C₃-C₈)alkynyl, (C₂-C₈)heteroalkyl and halo(C₁-C₈)alkyl, or two R¹⁷ groups attached to the same nitrogen atom are combined to form a five- to eight-membered ring.

11. A pharmaceutical composition of claim 10, wherein said compound is a compound of any of claims 2-9.

12. A method of modulating LXR function in a cell, said method comprising contacting said cell with an LXR-modulating amount of a compound of the formula: 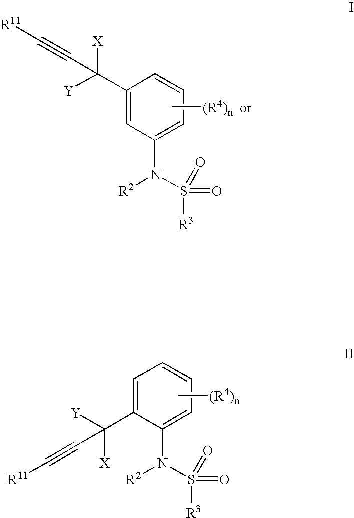
or a pharmaceutically acceptable salt or prodrug thereof, wherein
R¹¹ is a member selected from the group consisting of hydrogen, halogen, nitro, cyano, R¹², OR¹², SR¹², NHR¹², N(R¹²)₂, (C₅-C₈)cycloalkenyl, COR¹², CO₂R¹², CONHR², CON(R¹²)₂, C═N—NR¹², aryl(C₁-C₄)alkyl, heteroaryl, heteroaryl(C₁-C₄)alkyl, (C₄-C₈)cycloalkyl(C₁-C₄)alkyl and hetero(C₄-C₈)cycloalkyl(C₁-C₄)alkyl; wherein each R¹² is (C₁-C₈)alkyl, (C₃-C₈)alkenyl, (C₃-C₈)alkynyl, (C₂-C₈)heteroalkyl, halo(C₁-C₈)alkyl, (C₄-C₈)cycloalkyl, aryl or two R¹² groups attached to the same nitrogen atom are combined to form a five- to eight-membered ring and any alkyl portions of R¹¹ are optionally substituted with from one to three substituents independently selected from the group consisting of halogen, OR¹³, NHSO₂R¹⁴ and NHC(O)R¹³, and any aryl or heteroaryl portions of R¹¹ are optionally substituted with from one to five substituents independently selected from the group consisting of halogen, cyano, nitro, R¹⁴, OR¹³, SR¹³, N(R¹³)₂, CO₂R¹³, CON(R¹³)₂, C(O)R¹³, SO₂R¹³, SO₂N(R¹³)₂, NHSO₂R¹⁴, NHC(O)R¹³, phenyl, phenyl(C₁-C₈)alkyl and phenyl(C₂-C₈)heteroalkyl; wherein each R¹³ is independently selected from H, (C₁-C₈)alkyl, (C₃-C₈)alkenyl, (C₃-C₈)alkynyl, (C₂-C₈)heteroalkyl and halo(C₁-C₈)alkyl and each R¹⁴ is independently selected from (C₁-C₈)alkyl, (C₃-C₈)alkenyl, (C₃-C₈)alkynyl, (C₂-C₈)heteroalkyl and halo(C₁-C₈)alkyl;
X is a member selected from the group consisting of H, NH₂, NHR¹⁵, NHSO₂R¹⁵, OH and OR′, wherein R¹⁵ is (C₁-C₈)alkyl, (C₃-C₈)alkenyl, (C₃-C₈)alkynyl, (C₂-C₈)heteroalkyl or halo(C₁-C₈)alkyl and R′ is (C₁-C₈)alkyl, (C₃-C₈)alkenyl, (C₃-C₈)alkynyl, (C₂-C₈)heteroalkyl, halo(C₁-C₈)alkyl, aryl(C₁-C₄)alkyl, heterocyclo(C₅-C₈)alkyl, (C₁-C₄)alkylsulfonyl, arylsulfonyl, (C₁-C₄)alkylcarbonyl or (C₁-C₄)alkylsilyl;
Y is fluoro(C₁-C₄)alkyl;
R² is a member selected from the group consisting of H, (C₁-C₈)alkyl, (C₂-C₈)heteroalkyl, (C₃-C₈)alkenyl, (C₃-C₈)alkynyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino, or optionally, R² and R⁴ are combined to form a five- to seven-membered fused ring containing the nitrogen atom to which R² is attached and from 0 to 2 additional heteroatoms selected from N, O and S;
R³ is a member selected from the group consisting of aryl and heteroaryl, said aryl or heteroaryl group being optionally substituted with from one to five substituents independently selected from the group consisting of halogen, cyano, nitro, R¹⁶, OR¹⁶, SR¹⁶, COR¹⁶, CO₂R¹⁶, NHR¹⁶, N(R¹⁶)₂, CONHR¹⁶, CON(R¹⁶)₂, NHSO₂R¹⁶, NHC(O)R¹⁶, phenyl, phenyl(C₁-C₈)alkyl, and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring;
the subscript n is an integer of from 0 to 3; and
each R⁴ is independently selected from the group consisting of halogen, cyano, nitro, R¹⁷, OR¹⁷, SR¹⁷, COR¹⁷, CO₂R¹⁷, N(R¹⁷)₂ and CON(R¹⁷)₂, wherein each R¹⁷ is independently selected from H, (C₁-C₈)alkyl, (C₃-C₈)alkenyl, (C₃-C₈)alkynyl, (C₂-C₈)heteroalkyl and halo(C₁-C₈)alkyl, or two R¹⁷ groups attached to the same nitrogen atom are combined to form a five- to eight-membered ring.

13. A method of treating obesity, diabetes, hypercholesterolemia, atherosclerosis or hypolipoproteinemia, comprising administering to a subject in need thereof, a therapeutically effective amount of a compound of formula: 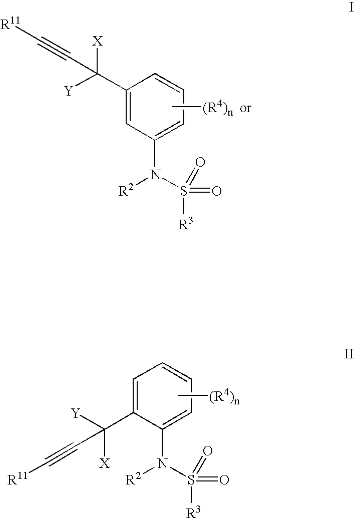
or a pharmaceutically acceptable salt or prodrug thereof, wherein
R¹¹ is a member selected from the group consisting of hydrogen, halogen, nitro, cyano, R¹², OR¹², SR¹², NHR¹², N(R¹²)₂, (C₅-C₈)cycloalkenyl, COR¹², CO₂R¹², CONHR¹², CON(R¹²)₂, C═N—NR¹², aryl(C₁-C₄)alkyl, heteroaryl, heteroaryl(C₁-C₄)alkyl, (C₄-C₈)cycloalkyl(C₁-C₄)alkyl and hetero(C₄-C₈)cycloalkyl(C₁-C₄)alkyl; wherein each R¹² is (C₁-C₈)alkyl, (C₃-C₈)alkenyl, (C₃-C₈)alkynyl, (C₂-C₈)heteroalkyl, halo(C₁-C₈)alkyl, (C₄-C₈)cycloalkyl, aryl or two R¹² groups attached to the same nitrogen atom are combined to form a five- to eight-membered ring and any alkyl portions of R¹¹ are optionally substituted with from one to three substituents independently selected from the group consisting of halogen, OR¹³, NHSO₂R¹⁴ and NHC(O)R¹³, and any aryl or heteroaryl portions of R¹¹ are optionally substituted with from one to five substituents independently selected from the group consisting of halogen, cyano, nitro, R¹⁴, OR¹³, SR¹³, N(R¹³)₂, CO₂R¹³, CON(R¹³)₂, C(O)R¹³, SO₂R¹³, SO₂N(R¹³)₂, NHSO₂R¹⁴, NHC(O)R¹³, phenyl, phenyl(C₁-C₈)alkyl and phenyl(C₂-C₈)heteroalkyl; wherein each R¹³ is independently selected from H, (C₁-C₈)alkyl, (C₃-C₈)alkenyl, (C₃-C₈)alkynyl, (C₂-C₈)heteroalkyl and halo(C₁-C₈)alkyl and each R¹⁴ is independently selected from (C₁-C₈)alkyl, (C₃-C₈)alkenyl, (C₃-C₈)alkynyl, (C₂-C₈)heteroalkyl and halo(C₁-C₈)alkyl;
X is a member selected from the group consisting of H, NH₂, NHR¹⁵, NHSO₂R¹⁵, OH and OR′, wherein R¹⁵ is (C₁-C₈)alkyl, (C₃-C₈)alkenyl, (C₃-C₈)alkynyl, (C₂-C₈)heteroalkyl or halo(C₁-C₈)alkyl and R′ is (C₁-C₈)alkyl, (C₃-C₈)alkenyl, (C₃-C₈)alkynyl, (C₂-C₈)heteroalkyl, halo(C₁-C₈)alkyl, aryl(C₁-C₄)alkyl, heterocyclo(C₅-C₈)alkyl, (C₁-C₄)alkylsulfonyl, arylsulfonyl, (C₁-C₄)alkylcarbonyl or (C₁-C₄)alkylsilyl;
Y is fluoro(C₁-C₄)alkyl;
R² is a member selected from the group consisting of H, (C₁-C₈)alkyl, (C₂-C₈)heteroalkyl, (C₃-C₈)alkenyl, (C₃-C₈)alkynyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino, or optionally, R² and R⁴ are combined to form a five- to seven-membered fused ring containing the nitrogen atom to which R² is attached and from 0 to 2 additional heteroatoms selected from N, O and S;
R³ is a member selected from the group consisting of aryl and heteroaryl, said aryl or heteroaryl group being optionally substituted with from one to five substituents independently selected from the group consisting of halogen, cyano, nitro, R¹⁶, OR¹⁶, SR¹⁶, COR¹⁶, CO₂R¹⁶, NHR¹⁶, N(R¹⁶)₂, CONHR¹⁶, CON(R¹⁶)₂, NHSO₂R¹⁶, NHC(O)R¹⁶, phenyl, phenyl(C₁-C₈)alkyl, and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring;
the subscript n is an integer of from 0 to 3; and
each R⁴ is independently selected from the group consisting of halogen, cyano, nitro, R¹⁷, OR¹⁷, SR¹⁷, COR¹⁷, CO₂R¹⁷, N(R¹⁷)₂ and CON(R¹⁷)₂, wherein each R¹⁷ is independently selected from H, (C₁-C₈)alkyl, (C₃-C₈)alkenyl, (C₃-C₈)alkynyl, (C₂-C₈)heteroalkyl and halo(C₁-C₈)alkyl, or two R¹⁷ groups attached to the same nitrogen atom are combined to form a five- to eight-membered ring.

14. A method of treating an LXR-mediated condition in a subject, said method comprising administering to said subject an LXR-modulating amount of a compound of the formula: 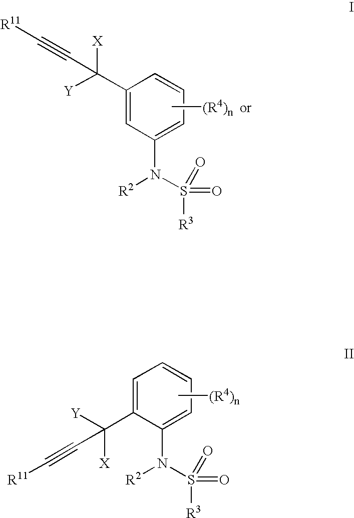
or a pharmaceutically acceptable salt or prodrug thereof, wherein
R¹¹ is a member selected from the group consisting of hydrogen, halogen, nitro, cyano, R¹², OR¹², SR¹², NHR¹², N(R¹²)₂, (C₅-C₈)cycloalkenyl, COR¹², CO₂R¹², CONHR¹², CON(R¹²)₂, C═N—NR¹², aryl(C₁-C₄)alkyl, heteroaryl, heteroaryl(C₁-C₄)alkyl, (C₄-C₈)cycloalkyl(C₁-C₄)alkyl and hetero(C₄-C₈)cycloalkyl(C₁-C₄)alkyl; wherein each R¹² is (C₁-C₈)alkyl, (C₃-C₈)alkenyl, (C₃-C₈)alkynyl, (C₂-C₈)heteroalkyl, halo(C₁-C₈)alkyl, (C₄-C₈)cycloalkyl, aryl or two R¹² groups attached to the same nitrogen atom are combined to form a five- to eight-membered ring and any alkyl portions of R¹¹ are optionally substituted with from one to three substituents independently selected from the group consisting of halogen, OR¹³, NHSO₂R¹⁴ and NHC(O)R¹³, and any aryl or heteroaryl portions of R¹¹ are optionally substituted with from one to five substituents independently selected from the group consisting of halogen, cyano, nitro, R¹⁴, OR¹³, SR¹³, N(R¹³)₂, CO₂R¹³, CON(R¹³)₂, C(O)R¹³, SO₂R¹³, SO₂N(R¹³)₂, NHSO₂R¹⁴, NHC(O)R¹³, phenyl, phenyl(C₁-C₈)alkyl and phenyl(C₂-C₈)heteroalkyl; wherein each R¹³ is independently selected from H, (C₁-C₈)alkyl, (C₃-C₈)alkenyl, (C₃-C₈)alkynyl, (C₂-C₈)heteroalkyl and halo(C₁-C₈)alkyl and each R¹⁴ is independently selected from (C₁-C₈)alkyl, (C₃-C₈)alkenyl, (C₃-C₈)alkynyl, (C₂-C₈)heteroalkyl and halo(C₁-C₈)alkyl;
X is a member selected from the group consisting of H, NH₂, NHR¹⁵, NHSO₂R¹⁵, OH and OR′, wherein R¹⁵ is (C₁-C₈)alkyl, (C₃-C₈)alkenyl, (C₃-C₈)alkynyl, (C₂-C₈)heteroalkyl or halo(C₁-C₈)alkyl and R′ is (C₁-C₈)alkyl, (C₃-C₈)alkenyl, (C₃-C₈)alkynyl, (C₂-C₈)heteroalkyl, halo(C₁-C₈)alkyl, aryl(C₁-C₄)alkyl, heterocyclo(C₅-C₈)alkyl, (C₁-C₄)alkylsulfonyl, arylsulfonyl, (C₁-C₄)alkylcarbonyl or (C₁-C₄)alkylsilyl;
Y is fluoro(C₁-C₄)alkyl;
R² is a member selected from the group consisting of H, (C₁-C₈)alkyl, (C₂-C₈)heteroalkyl, (C₃-C₈)alkenyl, (C₃-C₈)alkynyl, (C₃-C₈)cycloalkyl and (C₄-C₈)cycloalkyl-alkyl, wherein any alkyl portions of R² are optionally substituted with from one to three substituents independently selected from halogen, nitro, cyano, hydroxy, oxo and amino, or optionally, R² and R⁴ are combined to form a five- to seven-membered fused ring containing the nitrogen atom to which R² is attached and from 0 to 2 additional heteroatoms selected from N, O and S;
R³ is a member selected from the group consisting of aryl and heteroaryl, said aryl or heteroaryl group being optionally substituted with from one to five substituents independently selected from the group consisting of halogen, cyano, nitro, R¹⁶, OR¹⁶, SR¹⁶, COR¹⁶, CO₂R¹⁶, NHR¹⁶, N(R¹⁶)₂, CONHR¹⁶, CON(R¹⁶)₂, NHSO₂R¹⁶, NHC(O)R¹⁶, phenyl, phenyl(C₁-C₈)alkyl, and phenyl(C₂-C₈)heteroalkyl; wherein each R¹⁶ is independently selected from (C₁-C₈)alkyl, (C₃-C₈)alkenyl, (C₃-C₈)alkynyl, (C₂-C₈)heteroalkyl and halo(C₁-C₈)alkyl, or two R¹⁶ groups attached to the same nitrogen atom are combined to form a five- to eight-membered ring;
the subscript n is an integer of from 0 to 3; and
each R⁴ is independently selected from the group consisting of halogen, cyano, nitro, R¹⁷, OR¹⁷, SR¹⁷, COR¹⁷, CO₂R¹⁷, N(R¹⁷)₂ and CON(R¹⁷)₂, wherein each R¹⁷ is independently selected from H, (C₁-C₈)alkyl, (C₃-C₈)alkenyl, (C₃-C₈)alkynyl, (C₂-C₈)heteroalkyl and halo(C₁-C₈)alkyl, or two R¹⁷ groups attached to the same nitrogen atom are combined to form a five- to eight-membered ring.

15. A method in accordance with claim 14, wherein said condition is selected from the group consisting of obesity, diabetes, hypercholesterolemia, atherosclerosis and hyperlipoproteinemia.

16. A method in accordance with claim 15, wherein said compound is administered in combination with an anti-hypercholesterolemic agent.

17. A method in accordance with claim 14, wherein said compound is an LXR agonist.

18. A method of treating a condition selected from the group consisting of hypercholesterolemia, atherosclerosis and hyperlipoproteinemia, comprising administering to a subject in need thereof, a therapeutically effective amount of a compound of claim 1.

19. A method of treating a condition selected from the group consisting of diabetes and obesity, comprising administering to a subject in need thereof, a therapeutically effective amount of a compound of claim 1.
